(19) United States
(12) Patent Application Publication
(10) Pub. No.: US 2025/0375116 A1
(43) Pub. Date: Dec. 11, 2025

(54) SYSTEMS, DEVICES, AND METHODS FOR IMPROVING DECISION-MAKING OF IMPLANTABLE DEVICES USING MULTIPLE DATA SOURCES

(71) Applicant: North American EP Technology LLC, Edina, MN (US)

(72) Inventors: Robert Joseph GASKILL, Edina, MN (US); Yanhui LI, Edina, MN (US)

(73) Assignee: North American EP Technology LLC, Edina, MN (US)

(21) Appl. No.: 19/300,128

(22) Filed: Aug. 14, 2025

Related U.S. Application Data
(63) Continuation of application No. PCT/US2024/042740, filed on Aug. 16, 2024.
Application No. PCT/US2024/042740 is a continuation-in-part of application No. 18/529,544, filed on Dec. 5, 2023, now Pat. No. 12,337,184.
(60) Provisional application No. 63/533,062, filed on Aug. 16, 2023.
(60) Provisional application No. 63/533,062, filed on Aug. 16, 2023.
(60) Provisional application No. 63/566,807, filed on Mar. 18, 2024.

(51) Int. Cl. A61B 5/0215; A61B 5/00; A61N 1/05; A61N 1/365; A61N 1/39; G16H 50/30

(52) U.S. Cl. CPC A61B 5/02158; A61B 5/686; A61B 5/7264; A61B 5/746; A61N 1/0563; A61N 1/36564; A61N 1/3925; A61N 1/39622; G16H 50/30

(57) ABSTRACT

A method for assessing a physiological status of a patient is described herein. The method includes placing at least one pressure sensor in an anterior mediastinal space of the patient. Pressure signal data received from the at least one pressure sensor is amplified and filtered to separate the pressure signal data into different frequency bands. A set of pressure curves is generated based on the separated pressure signals in different frequency bands, and a physiological status of the patient is assessed based at least in part on the set of pressure curves.

### CROSS-REFERENCE TO RELATED APPLICATIONS

This application is a continuation of International Patent Application No. PCT/US2024/042740, filed Aug. 16, 2024, entitled “Systems, Devices, and Methods for Improving Diagnostic Predictions Using Multiple Data Sources,” which claims priority to, as a continuation-in-part, and the benefit of U.S. patent application Ser. No. 18/529,544 (now U.S. Pat. No. 12,337,184), filed Dec. 5, 2023, entitled, “Systems, Devices, and Methods for Improving Patient Outcomes in Implantable Cardioverter Defibrillators,” which claims priority to and the benefit of U.S. Provisional Patent Application No. 63/533,062, filed Aug. 16, 2023, entitled “Systems, Devices, and Methods for Improving Patient Outcomes in Implantable Cardioverter Defibrillators;” International Patent Application No. PCT/US2024/042740 also claims priority to and the benefit of U.S. Provisional Patent Application No. 63/566,807, filed Mar. 18, 2024, entitled “Systems, Devices, and Methods for Improving Diagnostic Predictions Using Multiple Data Sources,” and U.S. Provisional Patent Application No. 63/533,062, filed Aug. 16, 2023, entitled “Systems, Devices, and Methods for Improving Patient Outcomes in Implantable Cardioverter Defibrillators,” the disclosure of each of which is incorporated herein by reference in its entirety.

### BACKGROUND

The embodiments described herein relate generally to implantable treatment and/or diagnostic devices and more particularly, to systems, devices, and methods for improving treatment and/or diagnostic decision-making using multiple data sources.

Sensors are often used to measure certain characteristics associated with a patient. The characteristics and/or data indicative of or associated with the characteristics can be used for diagnosing various diseases, health events, conditions, and/or injuries of a patient, informing decision-making in providing treatment, and/or the like. However, in some instances, sensors can generate signals that may lead to false positives due to incorrect sensor placement, noise, and/or the like. The sensor can also generate signals that lead to false positives if the signal is indicating an anomaly in a characteristic associated with the patient that is typically associated with a known diagnosis or pathology, but the anomaly is actually caused by and/or associated with something other than what is being diagnosed (i.e., a false positive).

For example, the human heart is a mechanical pump for moving blood through the body and is driven by cardiac electrical activities. It therefore follows that cardiac electrical abnormalities (cardiac electrical signals) can result in abnormalities in the functioning of the mechanical pump, which in turn, may hinder the ability of the heart to move blood through the body. Moreover, abnormal heart function such as sudden cardiac arrest, arrhythmias, and/or the like can lead to sudden cardiac death.

Some known devices use one or more sensors configured to detect cardiac electrical signals. In some instances, the electrical signals may be associated with or indicative of arrythmia, ventricular tachycardia, ventricular fibrillation, atrial fibrillation, etc., but such signals may be influenced by other physiological and/or pathophysiological states. In some instances, such signals may accurately characterize an electrical state of the heart, but the state may resolve on its own after a relatively short time, or such an electrical state of the heart may not be associated with, may not coincide with, and/or may not cause an expected disfunction in the mechanical state of the heart (e.g., hemodynamic output). In such instances, relying on these signals alone may lead to an inaccurate or incomplete understanding of the actual state of the heart and/or false positive diagnoses of a health event, condition, disease state, etc.

False positives at the diagnostic stage, in turn, can lead to false and/or inappropriate treatments being provided to a patient. For example, an implantable cardioverter defibrillator (ICD) is a medical device that is designed to address and/or treat cardiac arrhythmias, heart failure, and other cardiac events that can lead to sudden cardiac death. The Cardiac Arrhythmia Suppression Trial (CAST) demonstrated that clinical benefit evaluation needs a desirable clinical end point of related mortality (survival). As a surrogate endpoint, ventricular premature heart beats do not provide a good evaluation of clinical benefit for patient outcomes. In clinical practice, making an optimum decision to manage an arrhythmia focuses not only on electrocardiogram (ECG) performance and diagnosis, but also on the patient's hemodynamic status and other clinical conditions. In general, however, the primary determinant(s) of shock therapy decisions in known ICDs is/are sensed cardiac electrical signals and/or derivations thereof such as, for example, heart rate, voltage, P wave, QRS morphology, ST segment, T wave, ECG diagnosis, and/or the like. In a patient with an ICD, a false positive diagnosis of ventricular fibrillation may cause the ICD to provide inappropriate defibrillation shock treatment, which can be painful, potentially dangerous, and has been shown to increase all-cause mortality. False positives may also cause patients and/or physicians to lose confidence with a diagnostic/treatment system, which can lead to improper tuning and/or adjusting (e.g., to decrease sensitivity and/or otherwise reduce undesired treatments), or to non-use of the diagnostic/treatment system. In such instances, actual health events, conditions, and/or diseased states (i.e., true positives) may be missed, which can be dangerous or even deadly for a patient.

In some known diagnostic and/or treatment systems, leads and/or sensors are often delivered into the heart transvenously, allowing the leads and/or sensors thereof to receive relatively clean ECG signals. While these ECG signals are clean (or at least substantially clean), challenges remain in the discrimination of true ventricular rate due to the potential confounding of multiple sources of events that can get classified as high ventricular rates in the ECG signals, without a corresponding or anticipated reduction of hemodynamic output. For example, an ECG signal associated with or otherwise suggesting atrial fibrillation may be classified by the diagnostic and/or treatment system as ventricular tachycardia or an ECG signal associated with or otherwise suggesting noise may be classified as ventricular fibrillation. These misclassifications can lead to misdiagnosis and/or the delivery of inappropriate treatment.

Furthermore, transvenous delivery of traditional leads/sensors and/or the indwelling of foreign objects in the heart can result in patient complications. In an effort to mitigate such complications, subcutaneous and/or substernal diagnostic/treatment systems have been developed that use leads and/or sensing electrodes that are placed external to the heart (e.g., in close proximity to or in contact with the pericardial tissue). For example, subcutaneous ICD systems, such as the subcutaneous defibrillation system developed by Cameron Health, have been shown to reduce complication rates, but such systems generally lack the ability to treat spontaneous ventricular tachycardia with anti-tachycardia pacing (a clinically proven method to treat dangerous arrhythmias without a defibrillation shock). Some known substernal ICD systems are able to provide cardiac pacing, however, the pacing thresholds in some such systems compared to transvenously delivered systems may prohibit or limit the ability of subcutaneous ICDs to leverage anti-tachycardia pacing to treat spontaneous ventricular tachycardia without triggering a painful, high-energy shock that may be considered inappropriate under certain conditions. Moreover, relying on only cardiac electrical signals as the primary determinant of shock therapy decisions, whether using traditional or subcutaneous/substernal ICDs, can result in false positives that can lead to the ICD activating and delivering an unnecessary/inappropriate shock to the patient.

Thus, there is a need for improving the decision-making of implantable diagnostic/treatment systems by using multiple sources of data to decrease false positives and reduce patient complications.

### SUMMARY

In some embodiments, a method for assessing a physiological status of a patient includes placing at least one pressure sensor in an anterior mediastinal space of the patient. The method further includes amplifying and filtering pressure signal data received from the at least one pressure sensor to separate the pressure signal data into different frequency bands. A set of pressure curves is generated based on the separated pressure signals in the different frequency bands, and a physiological status of the patient is assessed based at least in part on the plurality of pressure curves.

In some embodiments, an apparatus for assessing a physiological status of a patient includes an electrical sensor and at least one pressure sensor that are configured to be disposed within a mediastinal space of the patient. The electrical sensor is configured to detect electrical signals radiating from a heart of the patient. An amplifier/filter is coupled to the at least one pressure sensor and is configured to amplify and filter pressure signal data received from the at least one pressure sensor into pressure signals in different frequency bands. The apparatus further includes a memory and a processor configured to execute instructions stored in the memory. The instructions stored in the memory are operable to cause the processor to (i) receive the pressure signals in the different frequency bands, (ii) generate at least one pressure curve based on the pressure signals in different frequency bands, and (iii) receive, from the electrical sensor, data associated with the electrical signals radiating from the heart. The instructions stored in the memory are further operable to cause the processor to correlate the at least one pressure curve to the data associated with the electrical signals, and determine a physiological status of the patient based on the correlation.

In some embodiments, a system for reducing undesired treatments provided to a heart of a patient includes a first sensor and a second sensor that are configured to be disposed in a substernal space of a patient. The first sensor is configured to measure electrical signals radiating from the heart of the patient. The second sensor is configured to measure pressure signals in the substernal space. The system further includes an implantable cardioverter defibrillator (ICD) configured to be implanted in the patient and to be in communication with each of the first sensor and the second sensor. The ICD includes a generator configured to generate treatment energy and a lead configured to deliver the treatment energy from the generator to the heart of the patient. The generator includes a memory and a processor configured to execute instructions stored in the memory. The instructions stored in the memory are operable to cause the processor to determine (i) a hemodynamic status of the heart based on the pressure in the substernal space and (ii) a cardiac status based at least in part on a correlation of data from the first sensor representing the electrical signals radiating from the heart and the hemodynamic status. The instructions stored in the memory are further operable to (i) deliver to the heart the treatment energy generated by the generator in response to determining that the cardiac status is associated with an adverse health event and (ii) withhold the delivery of the treatment energy in response to determining that the cardiac status is not associated with an adverse health event.

In some embodiments, a non-transitory processor-readable medium stores code representing instructions to be executed by a processor that cause the processor to receive a first signal and a second signal associated with at least one characteristic of a heart of a patient, define a suspected cardiac status based at least in part on data from the first signal, and confirm the cardiac status based on a correlation between the data from the first signal and data from the second signal. Responsive to confirming the cardiac status, the code further causes a generator of an implantable cardioverter defibrillator (ICD) implanted in the patient to generate treatment energy. The ICD includes a lead, which in turn, is configured to apply the treatment energy to the heart of the patient.

### BRIEF DESCRIPTION OF THE DRAWINGS

FIG. 1 is an illustration depicting an anterior view of a human thoracic cavity.

FIG. 2 is an illustration depicting a left side view of the thoracic cavity and showing mediastinal divisions.

FIG. 3 is a schematic illustration depicting a diagnostic/treatment device including one or more sensors and engaging with a patient according to an embodiment.

FIG. 4 is a schematic illustration depicting an implantable cardioverter defibrillator (ICD) engaging with a patient according to an embodiment.

FIG. 5 is a schematic illustration depicting an ICD engaging with a patient according to another embodiment.

FIG. 6 is a flowchart depicting a method for determining if a patient is having a health event and whether to apply a corresponding corrective action according to an embodiment.

FIG. 7 depicts a placement of an ICD lead in the chest of a patient according to an embodiment.

FIG. 8 is a schematic illustration depicting an ICD in communication with an external compute device via a network according to an embodiment.

FIG. 9 is a schematic illustration depicting a diagnostic/treatment system engaging with a patient, according to an embodiment.

FIG. 10 is a flowchart depicting a method for determining if a patient is having a health event, according to an embodiment.

FIG. 11 is a flowchart depicting a method for determining a diagnostic status of a patient, according to an embodiment.

FIG. 12 is a schematic illustration of a diagnostic/treatment device couplable to external amplifier/filters and sensors, according to an embodiment.

FIG. 13A is a schematic illustration of an algorithm, performed by the diagnostic/treatment device of FIG. 12, associated with processing measured pressures into different pressure curves.

FIG. 13B is a schematic illustration of an algorithm, performed by the diagnostic/treatment device of FIG. 12, associated with determining cardiac rhythm based on measured electrical signals.

FIG. 13C is a schematic illustration of an algorithm, performed by the diagnostic/treatment device of FIG. 12, associated with determining a physiological status of a patient based at least in part on the pressure curves (FIG. 13A) and cardiac rhythm (FIG. 13B).

FIG. 14A is a graph showing one or more relationships between electrical signal data and mediastinal pressure data while a patient is maintained in a normal sinus rhythm, according to an embodiment.

FIG. 14B is a graph showing one or more relationships between electrical signal data and mediastinal pressure data during an induced changed from normal sinus rhythm to ventricular fibrillation, according to an embodiment.

FIG. 15 is a flowchart depicting a method for assessing a physiological status of a patient based at least in part on pressure sensor data according to an embodiment.

### DETAILED DESCRIPTION

The embodiments described herein relate generally to systems, devices, and/or methods for improving treatment and/or diagnostic decision-making or predictions associated with implantable health-based monitoring systems through the use of multiple data sources. In some embodiments, a treatment and/or diagnostic system can include and/or can be in communication with any number of sensors and/or other data sources configured to detect or otherwise provide data associated with one or more characteristics of a patient. The one or more characteristics can include one or more physiologic or pathophysiologic states as well as characteristics that are not detected by the sensors, such as patient demographic and/or health data (e.g., age, genetic information, health records, etc.). The one or more characteristics (or the underlying data indicative thereof) can be aggregated, correlated, verified, confirmed, corroborated, etc., which in turn, can be used to improve diagnostic accuracy (e.g., reduce undesirable, inappropriate, and/or inaccurate diagnoses, treatments, etc.) by confirming that an indication and/or prediction of a health event, or the like, from one data source is occurring based on correlated data from other data sources.

In some embodiments, the diagnostic/treatment systems and/or methods described herein can be at least partially implemented in and/or can otherwise include an implantable cardiac treatment device (e.g., cardiac therapy device, defibrillator, implantable cardioverter defibrillator (ICD), cardiac resynchronization therapy defibrillator (CRT-D), etc.) configured to deliver treatment (shock therapy) based at least in part on one or more characteristics associated with a heart of a patient. Alternatively, the diagnostic/treatment systems and/or methods described herein can be at least partially implemented in and/or can otherwise include an implantable diagnostic device configured to make diagnostic determinations and/or predictions independent of whether a corresponding treatment is provided. It should be understood that the embodiments and methods described herein can be implemented in as a diagnostic system, a treatment system, a combined diagnostic/treatment system, etc.

In some embodiments, a diagnostic and/or treatment system can include and/or can be in communication with one or more sensors configured to detect at least two characteristics associated with the heart. For example, a first characteristic can be cardiac signals and/or one or more derivatives thereof, which can be determined based at least in part on data from one or more sensors (e.g., one or more sensors used for detecting cardiac electrical signals, referred to generally as an “ECG sensor”). In this manner, the first characteristic can be similar to and/or substantially the same as the cardiac electrical signals that are used in some known ICDs. As described above, using cardiac electrical signals alone to make diagnostic and/or treatment decisions can lead to false positives and/or undesirable, incorrect, and/or inappropriate detection and/or therapy decisions. Accordingly, the devices, systems, and/or methods described herein are configured to aggregate, combine, analyze, correlate, confirm, verify, and/or otherwise process the first characteristic, or data used to determine the first characteristic, with any other suitable characteristic(s) (or data) associated with the patient to determine, for example, if the heart is having an irregular and/or adverse event such as, for example, tachycardia, ventricular fibrillation, sudden cardiac arrest, etc. For example, such a process of correlating characteristics and/or data can include determining whether a cardiac rhythm seen in cardiac electrical signal data has an expected corresponding result in the hemodynamic status and/or output signal data. In such examples, if a potential irregular cardiac rhythm is detected without a corresponding irregular hemodynamic status and/or output a suspected or initially diagnosed cardiac state may not be confirmed or verified and/or a treatment such as shock therapy may not be delivered.

In some implementations, the other characteristic(s) and/or data can be, for example, hemodynamic status as measured by one or more sensors directly or indirectly. For example, one or more sensors can be a pressure sensor, transducer, etc. configured to measure changes in pressure. In some implementations, the sensor(s) can be configured to directly measure and/or detect a hemodynamic status or a pressure associated with the hemodynamic status (e.g., blood pressure). In some implementations, the sensor(s) can be configured to indirectly measure and/or detect a hemodynamic status or a pressure associated with the hemodynamic status. For example, in some embodiments, a diagnostic/treatment system can include and/or can be in communication with one or more pressure sensors, transducers, and/or the like (referred to generally as “pressure sensor”), which is disposed in a substernal space (or anterior mediastinum) of the patient and in contact with and/or in close proximity to free wall of either the right, left, or both ventricles. In such embodiments, movement associated with the pumping/beating of the heart can result in pressure changes in the tissue or volumes surrounding the heart, which in turn, can be measured and/or detected by the pressure sensor. Similarly, the one or more pressure sensors can be configured to detect, sense, measure, etc. pressure changes associated with the movement and/or functioning of the lungs (i.e., respiration), a background mediastinal pressure, and/or the like.

In some implementations, inputs from the pressure sensor (and/or any other sensor) can be used and/or correlated with inputs of the ECG sensor to detect and/or determine a cardiac status and/or the occurrence of health events, such as arrhythmia, tachycardia, and/or the like. The cardiac status determined using the methods described herein can be more specific than determining cardiac status using cardiac electrical signal measurements alone, hemodynamic status measurements alone, and/or other cardiac characteristics individually. For example, because incorrectly withholding ICD therapy (false negative) is more harmful than incorrectly providing ICD therapy (false positive), current ICDs using only cardiac signal measurements (e.g., ECG signals) are typically designed with greater sensitivity and less specificity. Using a combination of cardiac signal measurements such as cardiac electrical signal measurements, cardiac mechanical signal measurements (e.g., hemodynamic rate, status, and/or output measurements), and/or other bio-signal measurements (e.g., pressure changes in the substernal space) can increase sensitivity and specificity, thereby reducing false results (false positives and false negatives). Additionally, detecting, sensing, and/or determining hemodynamic status can further confirm ventricular tachycardia, ventricular fibrillation, and/or other cardiac states. This results in specificity in ICD therapy decisions that is more beneficial to patients and supported by clinical evidence. In some implementations, cardiac pacing and/or shock treatment decisions can be made based on the signals from the one or more sensors (e.g., electrical signal measurements such as ECG signal measurements, mechanical signal measurements such as hemodynamic signal measurements, and/or any other bio-signal measurements such as respiratory signal measurements and/or the like) and/or the correlated data associated therewith (e.g., ex vivo data such as patient demographic and/or health data—age, weight, cardiac pathology, genetic information, health records, etc.).

In some embodiments, systems, devices, and/or methods described herein can be used, inter alia, to reduce undesired and/or inappropriate treatments provided by an ICD implanted in a patient (e.g., a transvenous ICD, subcutaneous ICD, and/or substernal ICD). For example, in some implementations, a method of providing treatment using an ICD can include receiving first signal data and second signal data and determining a cardiac status based at least in part on the first signal data and the second signal data. In some embodiments, the first signal data can be received from a first sensor and the second signal data can be received from a second sensor. In some embodiments, the first sensor can be configured to sense a first characteristic of the heart and the second sensor can be configured to sense a second characteristic of the heart, different from the first characteristic. Alternatively, the second sensor can be configured to sense a non-cardiac-related characteristic such as pressure changes due to respiration, and/or any other characteristic. The method further includes determining, based on the cardiac status, if a health event is occurring and, responsive to determining that a health event is occurring, applying a treatment to heart of the patient.

In some embodiments, a method for assessing a physiological status of a patient includes placing at least one pressure sensor in an anterior mediastinal space of the patient. The method further includes amplifying and filtering pressure signal data received from the at least one pressure sensor to separate the pressure signal data into different frequency bands. A set of pressure curves is generated based on the separated pressure signals in the different frequency bands, and a physiological status of the patient is assessed based at least in part on the plurality of pressure curves.

In some embodiments, an apparatus for assessing a physiological status of a patient includes an electrical sensor and at least one pressure sensor that are configured to be disposed within a mediastinal space of the patient. The electrical sensor is configured to detect electrical signals radiating from a heart of the patient. An amplifier/filter is coupled to the at least one pressure sensor and is configured to amplify and filter pressure signal data received from the at least one pressure sensor into pressure signals in different frequency bands. The apparatus further includes a memory and a processor configured to execute instructions stored in the memory. The instructions stored in the memory are operable to cause the processor to (i) receive the pressure signals in the different frequency bands, (ii) generate at least one pressure curve based on the pressure signals in different frequency bands, and (iii) receive, from the electrical sensor, data associated with the electrical signals radiating from the heart. The instructions stored in the memory are further operable to cause the processor to correlate the at least one pressure curve to the data associated with the electrical signals, and determine a physiological status of the patient based on the correlation.

In some embodiments, a system for reducing undesired treatments provided to a heart of a patient includes a first sensor and a second sensor that are configured to be disposed in a substernal space of a patient. The first sensor is configured to measure electrical signals radiating from the heart of the patient. The second sensor is configured to measure pressure signals in the substernal space. The system further includes an implantable cardioverter defibrillator (ICD) configured to be implanted in the patient and to be in communication with each of the first sensor and the second sensor. The ICD includes a generator configured to generate treatment energy and a lead configured to deliver the treatment energy from the generator to the heart of the patient. The generator includes a memory and a processor configured to execute instructions stored in the memory. The instructions stored in the memory are operable to cause the processor to determine (i) a hemodynamic status of the heart based on the pressure in the substernal space and (ii) a cardiac status based at least in part on a correlation of data from the first sensor representing the electrical signals radiating from the heart and the hemodynamic status. The instructions stored in the memory are further operable to (i) deliver to the heart the treatment energy generated by the generator in response to determining that the cardiac status is associated with an adverse health event and (ii) withhold the delivery of the treatment energy in response to determining that the cardiac status is not associated with an adverse health event.

In some embodiments, a non-transitory processor-readable medium stores code representing instructions to be executed by a processor that cause the processor to receive a first signal and a second signal associated with at least one characteristic of a heart of a patient, define a suspected cardiac status based at least in part on data from the first signal, and confirm the cardiac status based on a correlation between the data from the first signal and data from the second signal. Responsive to confirming the cardiac status, the code further causes a generator of an implantable cardioverter defibrillator (ICD) implanted in the patient to generate treatment energy. The ICD includes a lead, which in turn, is configured to apply the treatment energy to the heart of the patient.

In some embodiments, a system configured for reducing undesired treatments provided to a heart of a patient includes a first sensor configured to measure at least one characteristic of the heart of a patient and a second sensor configured to measure at least one characteristic of the heart different from the at least one characteristic measured by the first sensor. The system further includes an implantable cardioverter defibrillator (ICD) configured to be implanted in the patient. The ICD is in communication with each of the first sensor and the second sensor. The ICD includes a generator configured to generate treatment energy and a lead configured to deliver the treatment energy from the generator to the heart of the patient. The generator includes a memory and a processor configured to execute instructions stored in the memory. The instructions are operable to cause the processor to determine a cardiac status based at least in part on data from each of the first sensor and the second sensor, determine, based on the cardiac status, if a health event is occurring, and responsive to determining that the health event is an adverse health event, operate the generator to generate the treatment energy.

In some embodiments, a device configured for treating a patient includes a first sensor configured to measure at least one characteristic of a heart of the patient and a second sensor configured to measure at least one characteristic of the heart different from the at least one characteristic measured by the first sensor. The device further includes a lead configured to deliver treatment energy to the heart of the patient. The device further includes a generator operably coupled to the lead that is configured to generate the treatment energy. The generator includes a memory and a processor configured to execute instructions stored in the memory. The instructions are operable to cause the processor to receive a first signal from the first sensor and a second signal from the second sensor, determine a cardiac status based at least in part on the first signal and the second signal, determine, based on the cardiac status, if a health event is occurring, and responsive to determining that a health event is an adverse health event, operate the generator to generate the treatment energy.

In some embodiments, a non-transitory processor-readable medium stores code to cause the processor to receive a first signal and a second signal associated with at least one characteristic of a heart of a patient. The code causes the processor to determine a cardiac status based at least in part on the first signal and the second signal, and based on the cardiac status, determine if a health event is occurring. Responsive to the processor determining that an adverse health event is occurring, the code causes a generator of an implantable cardioverter defibrillator (ICD) implanted in the patient to generate treatment energy. The ICD, in turn, includes a lead configured to apply the treatment energy to the heart of the patient.

In some embodiments, a method for reducing undesired treatments provided by an implantable cardioverter defibrillator (ICD) implanted in a patient includes receiving a first signal and a second signal associated with at least one characteristic of a heart of the patient. A cardiac status is determined based at least in part on the first signal and the second signal. Based on the cardiac status, the method includes determining if an adverse health event is occurring, and responsive to determining that the adverse health event is occurring, applying a treatment to the heart of the patient via the ICD.

In some embodiments, a method for making health-based diagnostic predictions includes receiving sensor data from a plurality of sensors. The sensor data is associated with at least one characteristic of a patient. The method includes determining a status of the patient based on the sensor data. In some instances, the status can be associated with at least one of a physiological status of the patient and/or a pathophysiological status of the patient. A diagnostic state is determined based on the status, and a notification associated with the diagnostic status is generated.

In some implementations, the sensor data includes first data received from a first sensor from the plurality of sensors and second data received from a second sensor from the plurality of sensors. Determining the status (e.g., the physiological and/or pathophysiological status) may include correlating the first data and the second data. The first sensor may be different from the second sensor. The first sensor and the second sensor may be located in different positions in or on the patient. At least one of the first sensor or the second sensor may be implanted in a substernal or anterior mediastinum space of the patient. In some implementations, at least one sensor from the plurality of sensors is a sensor included in a wearable device.

In some implementations, the method further includes receiving patient information associated with the patient. The status (e.g., the physiological and/or pathophysiological status) may be determined based on the sensor data and the patient information.

In some embodiments, a diagnostic system may include one or more sensing device(s) that may be implanted in a patient to detect one or more characteristic. In addition or as an alternative, the diagnostic system may include one or more sensing device(s) that may be a wearable (e.g., smart watch, wrist cuff, ankle cuff, chest strap, smart ring, etc.). In some embodiments, the sensing device(s) may be configured to process, aggregate, correlate, confirm, verify, etc., data or may be configured to send data to an external or remote compute device(s) for processing. The sensing device(s) and/or compute device(s) may be configured to correlate one or more sensor reading to provide data, which in turn, may be used to diagnose and/or determine a physiological or pathophysiological state of a patient (e.g., determine if an acute medical event is occurring, evaluate health trends over time, and/or the like). In some embodiments, the sensing device(s) and/or the compute device(s) may be configured to generate a notification and/or an alarm system. The notification system can be configured to notify a user, patient, medical professional, emergency responder, and/or the like of a medical event and associated information. In some embodiments, the notification system can include and/or can be configured to provide one or more signals to an external and/or remote device such as a smartphone, a smart watch, and/or the like, which in turn, may provide a notification to the user (e.g., via a mobile application, etc.). In some embodiments, the notification system can include and/or can be configured to provide one or more signals that trigger an alarm (e.g., in a hospital or medical facility setting), place an emergency (911) call, etc.

In some embodiments, one or more machine learning models can be used for processing the data from the sensing device and providing, as an output, a predictive diagnosis. In some embodiments, various machine learning models can be used for processing data from any number of data sources, where each machine learning model is specifically trained to predict a health state/event based on a set or type of data (e.g., making a prediction based on cardiac electrical data, making a prediction based on cardiac mechanical data, making a prediction based on respiratory data, and/or the like). A different machine learning model can then be used for aggregation and/or correlation of the data and/or predictions from each machine learning model to determine (or at least corroborate) if a health event is occurring or for diagnosis.

The terminology used herein is for the purpose of describing particular embodiments, implementations, and/or concepts (including any feature(s) or aspect(s) thereof) and is not intended to be limiting. Unless defined otherwise, technical and/or scientific terms used herein have the same meanings as commonly understood by one of ordinary skill in the art. Any explanation or discussion of or using particular terms is intended to provide context and to facilitate understanding and is not necessarily intended to replace or supersede commonly used or known definitions understood by one skilled in the art unless explicitly stated otherwise. Moreover, various terms may be used to describe similar or substantially the same embodiments, implementations, and/or concepts (including any feature(s) or aspect(s) thereof) and thus, the use of particular term is not intended to be limiting and/or to the exclusion of other terms unless the terms are mutually exclusive, or the context clearly states otherwise.

As used herein, the singular forms “a,” “an,” and “the” are intended to include the plural forms as well, unless the context clearly indicates otherwise. With respect to the use of singular and/or plural terms herein, those having skill in the art can translate from the singular to the plurality and/or vice versa as is appropriate for the context and/or application. Furthermore, any reference herein to a singular component, feature, aspect, etc. is not intended to imply the exclusion of more than one such component, feature, aspect, etc. (and/or vice versa) unless expressly stated otherwise. The various singular/plural permutations may be expressly set forth herein for sake of clarity.

In general, terms used herein and in the appended claims are intended as “open” terms unless expressly stated otherwise. For example, the term “including” should be interpreted as “including but not limited to,” the term “having” should be interpreted as “having at least,” etc. Similarly, the term “comprising” may specify the presence of stated features, elements, components, integers (or fractions thereof), steps, operations, and/or the like but does not preclude the presence or addition of one or more other features, elements, components, integers (or fractions thereof), steps, operations, elements, components, and/or groups thereof, and/or the like unless such combinations are otherwise mutually exclusive.

As used herein the term “and/or” includes any and all combinations of one or more of the associated listed items. It should be understood that any suitable disjunctive word and/or phrase presenting two or more alternative terms, whether in the written description or claims, contemplates the possibilities of including one of the terms, either of the terms, or both/all of the terms. For example, the phrase “A and/or B” will be understood to include the possibilities of “A” alone, “B” alone, or a combination of “A and B.”

All ranges described herein include each individual member or value and are intended to encompass any and all possible subranges and/or combinations of subranges thereof unless expressly stated otherwise. Any listed range should be recognized as sufficiently describing and enabling the same range being broken down into at least equal subparts unless expressly stated otherwise.

As used herein, the terms “about,” “approximately,” and/or “substantially” when used in connection with stated value(s) and/or geometric structure(s) or relationship(s) is intended to convey that the value or characteristic so defined is nominally the value stated or characteristic described. In some instances, the terms “about,” “approximately,” and/or “substantially” can generally mean and/or can generally contemplate a value or characteristic stated within a desirable tolerance (e.g., plus or minus 10% of the value or characteristic stated). For example, a value of about 0.01 can include 0.009 and 0.011, a value of about 0.5 can include 0.45 and 0.55, a value of about 10 can include 9 to 11, and a value of about 1000 can include 900 to 1100. Similarly, a first surface may be described as being substantially parallel to a second surface when the surfaces are nominally parallel. While a value, structure, and/or relationship stated may be desirable, it should be understood that some variance may occur as a result of, for example, manufacturing tolerances or other practical considerations (such as, for example, the pressure or force applied through a portion of a device, conduit, lumen, etc.). Accordingly, the terms “about,” “approximately,” and/or “substantially” can be used herein to account for such tolerances and/or considerations.

As used herein, the words “proximal” and “distal” refer to a direction and/or position relative to a reference. The words “proximal” or “distal” can be relative terms and do not necessarily refer to universally fixed directions or positions. For example, in the context of a device that is manipulated by a user to engage a body of a patient, the terms “proximal” and “distal” generally refer to a direction and/or position that is closer to and away from, respectively, the user who would place the device into contact or engagement with the patient. Similarly state, an end or end portion of a device first touching the body of the patient would be the distal end or distal end portion, while the opposite end or end portion of the device (e.g., the end or end portion of the device being manipulated by the user) would be the proximal end or proximal end portion of the device. In the context of a device implanted in the body of a patient, an end or end portion of the device that is closer to the heart of the patient would be the distal end or distal end portion, while the opposite end or end portion (e.g., the end or end portion further from the heart) would be the proximal end or proximal end portion of the device.

As used herein, the term “characteristic(s)” described in reference to a patient generally refers to information associated with physiological and/or pathophysiological bio-signals of the patient. Such signals can be, for example, electrical signals, non-electrical (e.g., mechanical) signals, temperature-related signals, chemical or composition-related signals, and/or the like. Electrical signals can include any suitable signals associated with and/or otherwise indicative of the electrical functioning of the heart. The embodiments and/or methods described herein generally include implanting leads and/or sensors thereof in the anterior mediastinum of the patient, which can detect such electrical signals radiating from the heart. The detection and/or measurement of such cardiac electrical signals may include, but is not limited to, heart rate, voltage, P wave, QRS morphology, ST segment, T wave, ECG diagnosis, and/or the like, sensed through any suitable number of vectors. In some implementations, the characteristics and/or information or data indicative thereof can include, but are not limited to, cardiac or non-cardiac information and/or bio-measurements such as cardiac electrical signals, cardiac mechanical signals, and/or signals associated with cardiac cycle, pulmonary cycle, nervous system, body temperature, glucose level, pressure characteristics (e.g., blood pressure, pressure in the tissue or volumes surrounding the heart such as in the mediastinum, venous pressures, arterial pressures, and/or changes in such pressures, etc.), hemodynamic characteristics, volumetric characteristics (e.g., blood volume in the circulation system and/or changes thereof such as those detected via photoplethysmography (PPG)), oxygen saturation, sensed mechanical heart movement, cardiac sounds, cardiac echogram (ultrasound), cardiac Doppler, sleep performance, sleep apnea, recovery status, hemodynamic status, activity level, heart rate, respiratory rate, heart failure, and/or the like), data from internal or implanted medical devices (e.g., a pacemaker, an ICD, a CRT-D, ventricular assist device (VAD), a prosthetic device such as a heart valve prosthesis, etc.), and/or the like.

In some embodiments, the diagnostic/treatment systems and/or methods described herein can be at least partially implemented in and/or can otherwise include a treatment device such as a cardiac therapy device, ICD, CRT-D, implanted electric stimulator, pacemaker, etc. In some implementations, such devices can be used to deliver low-energy impulses used for cardiac pacing (a clinically proven method to treat dangerous arrhythmias without a defibrillation shock). As used herein, “cardiac pacing” or simply “pacing” generally refers to delivering low-energy impulses to “pace” the heart in response any suitable type of arrythmia. Examples of cardiac pacing can include, but are not limited to, anti-tachycardia pacing, bradycardia pacing, post-shock pacing, and/or the like. Anti-tachycardia pacing can include, for example, pacing shocks that provide therapy for ventricular tachycardia (e.g., a heartbeat that is faster than desired). Anti-bradycardia pacing can include, for example, pacing shocks that provide therapy for bradycardia (e.g., a heartbeat that is slower than desired). Post-shock pacing can include, for example, lower energy pacing that is delivered after a higher-energy shock is delivered (e.g., to facilitate the return of the heart to a normal sinus rhythm). Thus, while specific types arrythmias and/or specific types of pacing are described herein, it should be understood that they are presented by way of example only. The embodiments and/or methods described herein, inter alia, can be used to provide cardiac pacing to treat other types of arrythmias that may not be explicitly described herein.

The embodiments described herein and/or portions thereof can be formed or constructed of one or more biocompatible materials. In some embodiments, the biocompatible materials can be selected based on one or more properties of the constituent material such as, for example, stiffness, toughness, durometer, bioreactivity, etc. Examples of suitable biocompatible materials include but are not necessarily limited to metals, glasses, ceramics, and/or polymers. Examples of suitable metals include pharmaceutical grade stainless steel, gold, titanium, nickel, iron, platinum, tin, chromium, copper, and/or alloys thereof. A polymer material may be biodegradable or non-biodegradable. Examples of suitable biodegradable polymers include polylactides, polyglycolides, polylactide-co-glycolides, polyanhydrides, polyorthoesters, polyetheresters, polycaprolactones, polyesteramides, poly(butyric acid), poly(valeric acid), polyurethanes, biodegradable polyamides (nylons), and/or blends and copolymers thereof. Examples of non-biodegradable polymers include non-degradable polyamides (nylons), polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, and/or blends and copolymers thereof.

Non-limiting examples of suitable biocompatible polymer materials can include polylactides, polyglycolides, polylactide-co-glycolides, polyethylene-glycols, polyanhydrides, polyorthoesters, polyetheresters, polycaprolactones, polyesteramides, poly(butyric acid), poly(valeric acid), polyurethanes, polyamides (nylons), polyesters, polycarbonates, polyacrylates, polystyrenes, polypropylenes, polyethylenes, polyethylene oxide, polyolefins, polyethersulphones, polysulphones, polyvinylpyrrolidones, polyvinyl chloride, polyvinyl fluoride, poly(vinyl imidazole), polyether urethanes, silicone polyether urethanes, polyetheretherketones (PEEK), polytetrafluoroethylenes (PTFE), polylactones, chlorosulphonate polyolefins, ethylene-vinyl acetates and other acyl substituted cellulose acetates, elastomers, thermoplastics, and/or blends and copolymers thereof.

The embodiments, methods, and/or implementations herein, and/or the various features or advantageous details thereof, are explained more fully with reference to the non-limiting examples illustrated in the accompanying drawings and detailed in the following description. The examples and/or embodiments described herein are intended to facilitate an understanding of structures, functions, and/or aspects of the embodiments, ways in which the embodiments may be practiced, and/or to further enable those skilled in the art to practice the embodiments herein. Similarly, methods and/or ways of using or implementing the embodiments described herein are provided by way of example only and not limitation. Specific uses and/or implementations described herein are not provided to the exclusion of other uses unless the context expressly states otherwise. Descriptions of well-known components, methods, techniques, etc. may be omitted so as to not obscure the embodiments herein. Like numbers refer to like elements throughout.

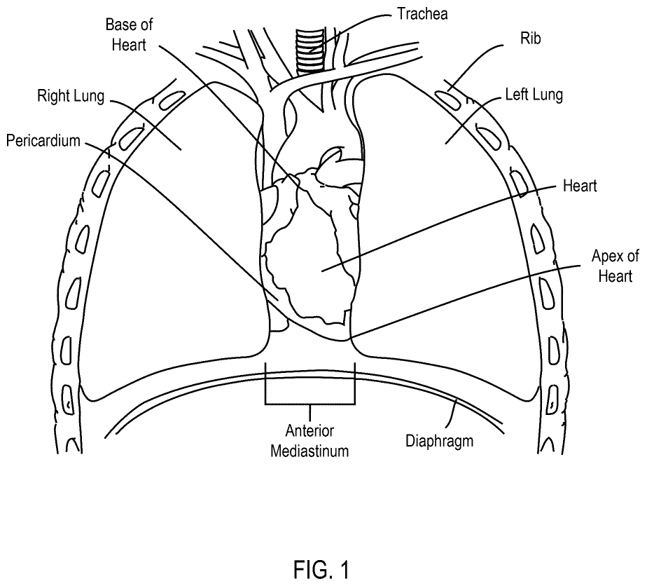

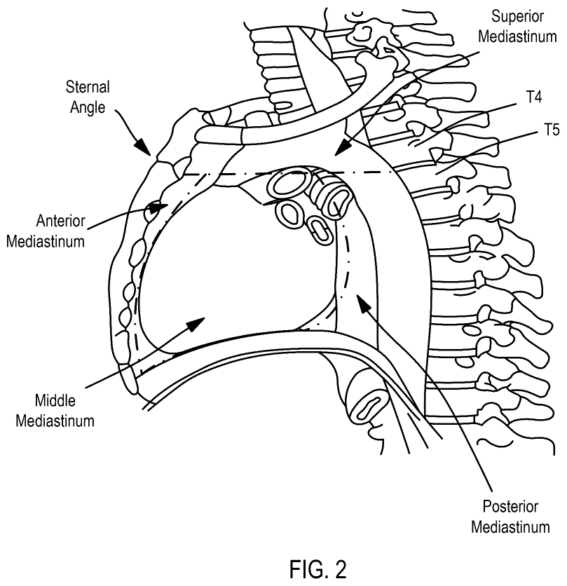

Referring now to the drawings, FIGS. 1 and 2 are different views of the human thoracic cavity and are shown to provide reference and context for the discussion of the various embodiments described herein. Specifically, FIG. 1 is an anterior view of a human thoracic cavity which illustrates various organs and structures. The trachea is a tube that connects a larynx to a left lung and a right lung, and allows air passage during respiration. The right and left lungs are shown, which are involved in the respiratory process of gas exchange. A pericardium is a doubled-walled sac including a heart bass of main blood vessels. The pericardium is an outer layer of cardiac tissue that provides protection and reduces friction during heartbeats. A first rib is shown, which is part of a ribcage. The ribcage provides structural support and protection for thoracic organs. The heart is shown, which pumps blood throughout a human body. Specifically, the heart receives oxygen-depleted blood from the body, via the patient's veins, and pumps the oxygen-depleted blood to the right and left lungs. The heart also receives oxygen-rich blood from the lungs, and pumps the oxygen-rich blood to the rest of the body via the patient's arteries. A base of the heart and an apex of the heart are shown, which are reference points of the heart. A diaphragm is also shown, which is a large, dome-shaped muscle at the base of the lungs which plays a role in breathing by contracting and expanding the thoracic cavity. The anterior mediastinum is shown, which is located in a central part of the thoracic cavity, above the pericardium. The anterior mediastinum is in front of the heart and pericardium but behind a sternum (not shown but foremost over the heart). The anterior mediastinum approximately extends from below the trachea vertically down to the diaphragm. The anterior mediastinum plays a multitude of functions, including but not limited to providing structural support, acting as a cavity for a thymus, and providing a pathway for blood vessels, nerves, and lymphatics.

FIG. 2 is a sagittal cross-sectional view of the thoracic cavity of FIG. 1, showing the different compartments or divisions of the mediastinum and surrounding structures. T4 and T5 are shown, which are the fourth and fifth thoracic vertebrae, respectively, that form a portion of the patient's spine. A sternal angle is shown, which is a joint that serves as a landmark to locate a second rib of a ribcage and a level of an intervertebral disc between T4 and T5. A superior mediastinum is shown, which is a region that extends from a top of the thoracic cavity down to the sternal angle and includes structures like a trachea, esophagus, and major blood vessels. The anterior mediastinum is shown, between the sternum (the bones below the sternal angle) and the heart. A middle mediastinum is located centrally in the thoracic cavity and includes the heart and roots of main blood vessels (e.g., aortic artery, pulmonary vein, etc.). A posterior mediastinum is also shown, which is behind the heart and in front of the spine.

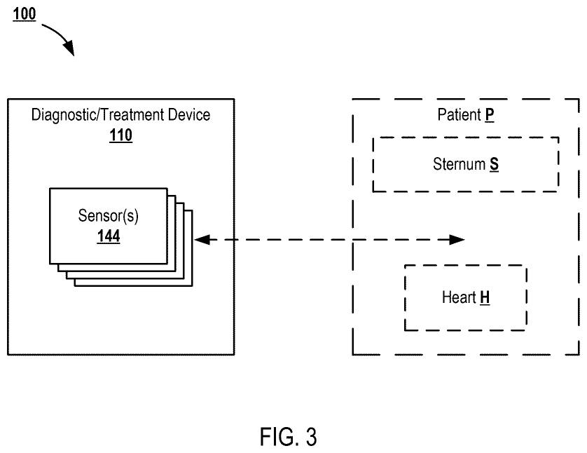

FIG. 3 is a schematic illustration of a diagnostic/treatment system 100 according to an embodiment. The diagnostic/treatment system 100 (referred to herein as “system 100”) includes a diagnostic/treatment device 110 having and/or in communication with a set of sensors 144. The diagnostic/treatment device 110 is configured to be implanted in a patient P. For example, at least the set of sensors 144 of the diagnostic/treatment device 110 is placed or implanted between a heart H and a sternum S of the patient P.

In some implementations, the diagnostic/treatment device 110 can be permanently implanted or temporarily placed between the heart H and the sternum S. The diagnostic/treatment device 110 can be any suitable device configured to perform any number of diagnostic and/or treatment processes based at least in part on data received from the set of sensors 144. In some embodiments, for example, the diagnostic/treatment device 110 can be configured to analyze and/or process data (including, but not limited to, data from the set of sensors 144) to provide one or more diagnoses and/or diagnostic predictions associated with the health of the patient P. In some implementations, the diagnoses and/or diagnostic predictions can be associated with, for example, the health and/or functioning of the patient's heart, lungs, and/or other portions of the patient's body. In some embodiments, the diagnostic/treatment device 110 can be a device configured to provide treatment and/or therapy to one or more portions of the patient P (e.g., the heart H of the patient P). For example, the diagnostic/treatment device 110 can be an ICD, a CRT-D, a pacemaker, and/or any other suitable device. In such embodiments, the diagnostic/treatment device 110 can be configured to analyze and/or process data (including, but not limited to, data from the set of sensors 144) to inform and/or to make one or more decisions associated with providing treatment and/or therapy to the patient P. For example, in the case of an ICD, the diagnostic/treatment device 110 can be configured to determine whether to provide electric shock therapy (e.g., a defibrillation shock, cardiac pacing, and/or the like) to the heart H of the patient P based at least in part on data received from the set of sensors 144. In some embodiments, the diagnostic/treatment device 110 can be configured to provide and/or perform diagnostic as well as treatment functionality.

The set of sensors 144 can include any number of sensors configured to detect bio-signals and/or other signals associated with a patient. For example, the set of sensors 144 can include one or more sensors implanted in the body and configured to detect and/or measure one or more characteristics and/or signals associated with the cardiovascular system, the respiratory system, and/or any other suitable system or portion of the body. In some embodiments, one or more sensors can be disposed outside of the body (e.g., included in a wearable such as a smartwatch, fitness tracker, an insulin pump, a thermometer, a pulse oximeter, a smart ring, and/or the like). Examples of sensors 144 include, but are not limited to, a sensing lead, a pressure sensor, a photoplethysmography sensor (PPG) sensor (or other optical sensor), an oxygen saturation (SpO2) sensor, an electrocardiogram (or cardio electrogram) sensor, an accelerometer, a temperature sensor, an acoustic sensor, an ultrasound sensor, and/or the like. In some embodiments, the sensors 144 can be configured to monitor and/or measure multiple characteristics associated with the patient P, which in turn, can be used to determine and/or define one or more treatment decisions, diagnoses, diagnostic predictions and/or the like. As described in detail herein, the data associated with and/or indicative of the multiple characteristics can be correlated, aggregated, confirmed, verified, etc. to allow for more accurate and precise diagnostic and/or treatment decisions than a diagnostic and/or treatment decision using just one characteristic.

A first sensor configured to detect and/or measure at least one characteristic of or associated with the heart H and a second sensor configured to detect and/or measure at least one characteristic of or associated with the heart H that is different from the characteristic(s) measured by the first sensor. The set of sensors may also include one or more sensors (e.g., a third sensor) configured to detect and/or measure a pressure in the space (e.g., portion of the body) in which the third sensor is placed. The diagnostic/treatment device 110 with the set of sensors may be configured to monitor, diagnose, and/or treat a patient's health (e.g., the device may be a diagnostic device only, a therapeutic device only, or a diagnostic and therapeutic device).

In some implementations, the coordination of the data obtained from the set of sensors 144 can be used to provide and/or determine a health status of the patient and/or to make or inform treatment decisions. This can be performed, for example, by selectively separating the data received from one or more sensors 144 as a function of the source, characteristic, and/or bio-signal being detected. For example, any suitable amplification and filtering (either digitally or through physical circuitry) can be performed on raw sensor data to separate the data into multiple signals, vectors, modalities, characteristics, etc. In some embodiments, the sensors 144 can include one or more pressure sensors that can sense and/or detect pressures and/or pressure changes in, for example, the anterior mediastinum. In such embodiments, the data can be separated based on the physiological and/or pathophysiological characteristic producing the pressure signal. For example, the pressure data can be separated (e.g., via amplification and/or filtering) into a respiratory pressure curve, a cardiac pressure curve, and a mediastinal pressure curve. In some embodiments, the amplification and/or filtering can be performed based at least in part on differing frequencies within the pressure data as described in detail below with reference to specific embodiments. Moreover, understanding the individual pressure curves associated with the physiologic and/or pathophysiologic cause of the pressure changes can allow for improved monitoring (and/or improved specificity of the collected or measured data), which in turn, can reduce the likelihood of false positive diagnoses as well as false positives in therapeutic/treatment decision-making, as described in further detail herein.

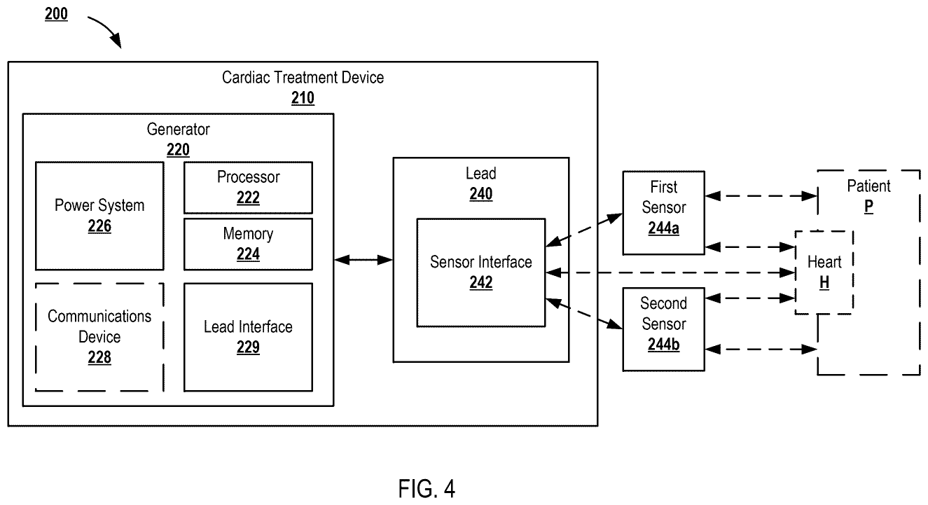

FIG. 4 schematically depicts diagnostic/treatment system 200 engaging a patient P according to an embodiment. In some embodiments, the diagnostic/treatment system 200 can be similar to and/or can be a specific implementation of the diagnostic/treatment system 100 described above with reference to FIG. 3. As shown, the diagnostic/treatment system 200 includes a cardiac treatment device 210 such as an ICD, a CRT-D, and/or the like. More particularly, in this embodiment, the cardiac treatment device 210 is an ICD implanted (or implantable) in the patient P. As described herein, the diagnostic/treatment system 200 can be utilized for treating certain conditions or states of a heart H of the patient P via certain electric treatment therapies while reducing complications and/or misdiagnoses that can lead to undesired, unnecessary, and/or inappropriate treatments (e.g., “false positives”).

The cardiac treatment device 210 includes a generator 220 and a lead 240. The diagnostic/treatment system 200 (also referred to herein as “system”) further includes a first sensor 244a and a second sensor 244b in communication with the cardiac treatment device 210. While the system 200 is described herein as including “the first sensor 244a” and “the second sensor 244b,” it should be understood that the first sensor 244a can be a single sensing device or multiple sensing devices that collectively function as the first sensor 244a, and similarly, the second sensor 244b can be a single sensing device or multiple sensing devices that collectively function as the second sensor 244b. In some implementations, multiple sensing devices can allow for sensor data that includes multiple signal vectors (e.g., multiple cardiac electrical and/or mechanical signal vectors). Similarly, while the system 200 is described herein as including “the first sensor 244a” and “the second sensor 244b,” it should be understood that the system 200 can include any number of additional sensors configured to sense and/or detect any suitable characteristic(s) associated with the patient (e.g., cardiac electrical and/or mechanical signals or any suitable non-cardiac signals). In some implementations, each sensor can be configured to sense and/or detect a different characteristic associated with the heart, or more generally, the patient. In some implementations, one or more sensors can be configured to sense or detect the same characteristic, thereby allowing for confirmation/verification of signal data and/or a desired degree of sensitivity and/or specificity in interpreting the signal data.

The cardiac treatment device 210 is configured to receive signals from the first sensor 244a and the second sensor 244b associated with one or more characteristics of the patient P or the heart H of the patient P. In some embodiments, the cardiac treatment device 210 can receive multiple signals from one or both of the first sensor 244a and the second sensor 244b. The cardiac treatment device 210 includes the generator 220, which can include a processor 222, a memory 224, a power system 226, a communication device 228, and a lead interface 229. The generator 220 is configured to determine when to provide treatment and to generate treatment energy that can be sent to the heart H via the lead 240. In some embodiments, the generator 220 can be placed on the pectoralis major muscle of the patient P, behind the pectoral major muscle, on the abdomen, along the left exterior thorax, or elsewhere on or in the body of the patient P.

The processor 222 is configured to execute the operations of the generator 220. The processor 222 can be, for example, a hardware based integrated circuit (IC), or any other suitable processing device configured to run and/or execute a set of instructions or code. For example, the processor 222 can be a general-purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC), and/or the like. The processor 222 can be operatively coupled to the memory 224 through a system bus (for example, address bus, data bus, and/or control bus).

The memory 224 stores instructions that are executed by the processor 222. The memory 224 can be any suitable volatile or non-volatile memory such as, for example, a random-access memory (RAM), a memory buffer, a hard drive, a read-only memory (ROM), an erasable programmable read-only memory (EPROM), a flash memory, and/or the like. In some instances, the memory 224 can store, for example, one or more software programs and/or code that can include instructions to cause the processor 222 to perform one or more processes, functions, and/or the like. In some implementations, the memory 224 can include extendable storage units that can be added and used incrementally. In some instances, the memory 224 can be remotely operatively coupled with a compute device (not shown). For example, a remote database device can serve as a memory (or at least a portion of a memory) and be operatively coupled to the compute device (e.g., via a network or the like).

The memory 224 can, for example, include instructions associated with processing data received from one or more data sources (e.g., sensors, etc.); determining, predicting, and/or diagnosing a state, status, and/or event (e.g., a physiological and/or pathophysiological state, status, and/or event); determining when treatment should be applied to the heart H of the patient P, and/or the like. The memory 224 can include instructions that can cause the processor 222 to analyze sensor data received from the first sensor 244a and/or the second sensor 244b and to determine and/or define one or more predictions, diagnoses, treatment characteristics, modalities, methods, etc. These instructions can be updated and/or refined based on learnings from previously received sensor data. The memory 224 can further include instructions for managing the power usage of the power system 226, which can be configured to apply or provide electrical power (e.g., shock) according to the treatment characteristics, modalities, methods, etc., determined and/or defined by the processor 222.

The memory 224 can, in some embodiments, include instructions associated with preprocessing the signals from the first sensor 244a and the second sensor 244b. For example, the memory 224 can include and/or store instructions that when executed by the processor 222 perform one or more preprocessing steps that can include amplification, filtering, analog to digital conversion, signal recognition and detection, synchronization, and/or the like. The processor 222 is configured to execute instructions, stored and/or included in the memory 224, for determining a status (e.g., a cardiac status) based on the signals, with or without preprocessing. The status (e.g., cardiac status) can be associated with a heartbeat, an output of the heart, bodily processes of the patient P, one or more systems of the body of the patient P, a portion of the body of the patient P, sensor data, and/or the like. Determining the status (e.g., cardiac status) can include comparing an output from the first sensor 244a and an output from the second sensor 244b to recognize and confirm the status (e.g., cardiac activity and/or the like). For example, if the output from the first sensor 244a appears to indicate an irregular heartbeat, but the output from the second sensor 244b does not appear to indicate an irregular heartbeat, then the output from the first sensor 244a may be determined to be a false positive. Determining and/or identifying false positives can allow the cardiac treatment device 210 to decrease the number of undesired treatments for the patient P and is more specific than if using an output from just one of the first sensor 244a or the second sensor 244b. If both the first sensor 244a and the second sensor 244b indicate the same, complementary, and/or verified cardiac status, that cardiac status is determined and/or confirmed. For example, in some embodiments, the memory 224 can include instructions associated with correlating heart rate data (e.g., from the first sensor 244a) and hemodynamic output or status data (e.g., from the second sensor 244b).

For example, in some embodiments, the memory 224 can include instructions associated with correlating a first output from the first sensor 244a and a second output from the second sensor 244b. In some embodiments, the first output and the second output can be complementary and confirmatory functions, characteristics, states, etc. For example, the first output can be an electrical characteristic (e.g., cardiac electrical data) and the second output can be a mechanical characteristic (e.g., mechanical data), which can allow the processor 222 to correlate, confirm, and/or verify an electrical characteristic with or to an expected mechanical characteristic. As another example, the first output can be respiratory data (e.g., respiratory rate, period, cycle, etc. as determined and/or sensed by a pressure sensor and/or the like), and the second output can be oxygen saturation data (e.g., from an internal or external pulse oximeter and/or the like). In some embodiments, the memory 224 can include instructions associated with correlating the outputs from the sensors 244a, 244b any other data associated with the patient P.

In some embodiments, determining the cardiac status can utilize an artificial intelligence algorithm (e.g., a machine learning model and/or algorithm) to further refine, determine, and/or predict the cardiac status. For example, the machine learning model can be trained to determine the cardiac status based at least in part on cardiac signal data and hemodynamic status data. In some embodiments, the machine learning model and/or algorithm (referred to herein as machine learning “model” or “algorithm” interchangeably) can be configured to filter out or recognize physiological activity (e.g., breathes, heartbeats, physical activity, etc.) that were determined or missed during an initial determination. In some embodiments, the machine learning algorithm can correlate the outputs from the first sensor 244a and the second sensor 244b to determine a pattern corresponding to a cardiac rate, status, QRS complex, etc. In some embodiments, the machine learning algorithm can learn from the outputs of the first sensor 244a and the second sensor 244b to determine long-term correlation and changes in correlation between the output of the first sensor 244a and the second sensor 244b.

The generator 220 (or processor 222 thereof) may be configured to determine a cardiac status based on the signals, with or without preprocessing. The cardiac status can be associated with a heartbeat, an output of the heart, sensor data, and/or the like. Determining the cardiac status can include comparing an output from sensor(s) to recognize and confirm cardiac activity. The generator 220 is further configured to determine if a health event (e.g., an adverse health event that may be undesirable or dangerous to the patient such as, for example, cardiac arrest, ventricular fibrillation, cardiac tamponade, tachycardia, and/or other cardiac electrical or mechanical pathologies) is occurring based on the cardiac status. The health event can correspond to at least one of cardiac attack, tachycardia, fibrillation, arrythmias, and/or the like.

In some embodiments, the processor 222 may execute a machine learning algorithm, which may be the same or different than that used in refining, determining, and/or predicting the cardiac status, for determining if a health event is occurring. The memory 224 can further include and/or store instructions for execution by the processor 222 associated with determining the type of health event. For example, the processor 222 determining the type of health event can include correlating the cardiac status with known cardiac statuses associated with health events. In some embodiments, the memory 224 can further include and/or store instructions for determining if the health event is an adverse health event. Similarly stated, the memory 224 can include and/or store instructions for determining and/or distinguishing an adverse health event from, for example, a normal or non-adverse health event that may present and/or may be associated with one or more similar characteristics.

In some embodiments, the processor 222 can determine the type of health event (e.g., any of the adverse health events described herein). In some embodiments, the processor 222 may execute one or more machine learning algorithms, which may be the same or different than the other machine learning algorithms described herein, for determining the type of health event. In some implementations, determining the type of health event can include correlating the cardiac status with known cardiac statuses associated with health events. Based on the type of health event determined, the memory 224 may store and/or include instructions for execution by the processor 222 associated with determining a treatment based on the type of health event. For example, the memory 224 may store and/or include instructions associated with determining the treatment based on the type of adverse health event. For example, if the adverse health event is determined to be tachycardia, then the treatment may correspond to anti-tachycardia pacing (e.g., low-energy shocks used to pace the heart to beat at a slower rate). As another example, if the health event is determined to be bradycardia, then the treatment can correspond to anti-bradycardia pacing (e.g., low-energy shocks used to pace the heart to beat at a faster rate). As another example, if the health event is determined to be heart failure, then the treatment may be heart failure treatment, which may include a high-energy shock, followed by low-energy shocks used to return the heart to a normal sinus rhythm (e.g., post-shock pacing).

The memory 224 can store and/or include instructions for execution by the processor 222 associated with determining the type of treatment as well as specific parameters for the treatment based on the adverse health event and, optionally, any other information (e.g., patient information and/or history, physiology, pathophysiology, cardiac status, sensor signals, etc.). The memory 224 further stores and/or includes instructions for execution by the processor 222 for applying the treatment to the heart H of the patient P. This can include instructions for commanding and/or at least partially controlling the cardiac treatment device 210 to deliver the treatment to the heart H of the patient P (e.g., via the power system 226). In some embodiments, the memory 224 can store and/or include instructions that cause the processor 222 to alter or pause treatment during treatment delivery based on outputs from the first sensor 244a and the second sensor 244b. In some embodiments, the memory 224 can store and/or include instructions that cause the processor 222 to operate the generator 220 to generate the treatment energy based on the type, severity, duration, and/or other characteristic(s) of an adverse health event.

The power system 226 is configured to store energy for use by the cardiac treatment device and to generate treatments for delivery to the patient P. In some embodiments, the power system 226 can include at least one battery (e.g., LiPo, Li-ion, etc.). The at least one battery can be charged when the battery is low on power. In some embodiments, the power system 226 includes a primary cell battery and a rechargeable battery. In some embodiments, the power system 226 can be configured to charge automatically (e.g., via patient P movement) or wirelessly (e.g., via inductive charging). The power system 226 can also be configured to generate a therapy signal. For example, the therapy signal can include treatment energy for at least one of cardiac pacing and/or defibrillation shock therapy. In some implementations, the treatment energy for cardiac pacing can be low-power or relatively low-power treatment energy and the treatment energy for shock therapy can be high-power or relatively high-power treatment energy. In some embodiments, the operation of the power system 226 is controlled via the processor 222 executing instructions stored in the memory 224. In some embodiments, the processor 222 may execute instructions that result in the power system 226 cycling the power of the first sensor 244a and/or the second sensor 244b to conserve power.

The communication device 228 is configured to allow the generator 220 to communicate with one or more devices such as, for example, an external device, an implantable device, controller, server, etc. (e.g., a compute device, controller, etc. of a medical professional, the patient P, and/or the like). The communication device 228 can be configured to send information via one or more networks using any suitable communication mode (e.g., Bluetooth, Wi-Fi, near field communication (NFC), and/or the like). The communication device 228 can be configured to send information with a wired or wireless communication device. In some embodiments, the communication device 228 may be optional.

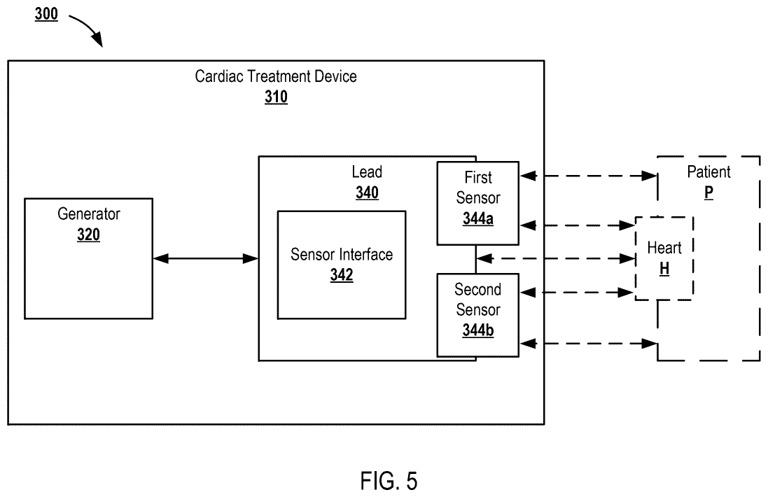

In some implementations, the communication device 228 can send information to a user and/or a compute device controlled by a user regarding the cardiac treatment device 210 such as battery level, information regarding therapies delivered to the heart H of the patient P, device status, and/or the like. The communication device 228 can also send information regarding the patient P, such as real-time or substantially real-time information, data, and/or other signals associated with the heart H (e.g., data from the first sensor 244a and/or the second sensor 244b, and/or any other suitable data). In some embodiments, the communication device 228 can receive signals for augmenting and/or at least partially controlling the operation of the cardiac treatment device 210. For example, the communication device 228 can receive signals (e.g., from a controller or external compute device) associated with patient information and/or other operational instructions for determining when the generator 220 generates a therapy, power levels associated with therapy, pacing and/or other thresholds (e.g., for anti-tachycardia pacing, bradycardia pacing, post-shock pacing, and/or any other suitable pacing therapy), and/or the like. In some embodiments, the communication device 228 can be configured to send information and/or signals to an external compute device and/or server that is/are configured to perform machine learning processes. The communication device 228 can also receive information from the external compute device and/or the server that may include instructions, firmware, updates, etc. based on an output from a machine learning model. An example of an external compute device is described in further detail in reference to FIG. 5.

The lead 240 is operatively coupled to the generator 220 via the lead interface 229. The lead interface 229 is configured to couple (e.g., physically couple or at least operably couple) the generator 220 to the lead 240 to allow data signals and/or electric power (e.g., for therapy) to be transferred therebetween. In some embodiments, the lead interface 229 can be configured to format data signals and/or electric power transferred between the generator 220 and the lead 240 into any suitable format allowing, for example, the processing of sensor data or the like by the generator 220 and/or the application of therapy by the lead 240. The lead interface 229 may be a flexible interface to provide flexibility for the continuous movement of the heart and allowing the lead 240 to maintain contact with the heart.

In some embodiments, the lead 240 can extend away from the generator 220 via a conduit that includes a signal/power carrying wire. The lead 240 is configured to deliver treatment to the patient P and/or the heart H of the patient P. As shown in FIG. 4, the lead 240 includes, is operably coupled to, and/or is in communication with the first sensor 244a and the second sensor 244b via a sensor interface 242. For example, the sensors 244a and/or 244b can be included and/or coupled along one or more portions of the lead 240. The sensor interface 242 is configured to communicate with the sensors 244a, 244b to send and/or receive signals between the sensors 244a and 244b, and the lead 240, which in turn is in communication with the generator 220. Alternatively, in some embodiments, the sensors 244a and 244b can be in communication with the lead interface 229 and/or otherwise can be directly in communication with the generator 220. Additionally, in some embodiments, the sensor interface 242 provides the first sensor 244a and the second sensor 244b with power. In some embodiments, the sensor interface 242 can preprocess signals output by and/or received from the first sensor 244a and the second sensor 244b. In some embodiments, the outputs of the sensor interface 242 and/or the first sensor 244a and/or the second sensor 244b can be calibrated based on the patient P, the position in the body of the patient P, and/or the like. For example, the first sensor 244a and/or the second sensor 244b can be calibrated based on one or more known measurement(s) of patient characteristic(s). Calibration can include calibrating the output of the sensors 244a and 244b to align with known measurements of patient characteristics. For example, a cardiac electrical signal sensor used to measure, for example, cardiac rate can be calibrated based on comparing and adjusting the sensor output to an external cardiac rate measuring device.

The lead 240 can include shocking elements, coils, electrodes, etc. for delivering treatment (e.g., including energy from the generator) to the heart H of the patient P. In some embodiments, the lead 240 is positioned under the sternum in the substernal space and/or anterior mediastinum of the patient P. In some embodiments, the lead 240 is positioned in contact with, adjacent to, and/or otherwise in close proximity to the fibrous pericardium of the heart H. In some embodiments, the lead 240 can be positioned endocardial, epicardial, or under the sternum. In some embodiments, the lead 240 can be designed and/or formed to use, traverse, and/or fill (or at least substantially use, traverse, and/or fill) at least a portion of the volume between the sternum and the pericardium of the heart H (e.g., the substernal space and/or the anterior mediastinum). Similarly stated, the lead 240 can be sized and/or shaped such that a first portion or first portions of the lead 240 is/are in contact with, for example, a posterior surface of the sternum and a second portion or second portions are in contact with, adjacent to, and/or otherwise in close proximity to the fibrous pericardium of the heart H. In some embodiments, the lead 240 can have a three-dimensional design allowing the lead 240 to use, traverse, and/or fill at least a portion of the volume between the sternum and the heart H (e.g., the substernal space and/or the anterior mediastinum). In some embodiments, the lead 240 can include any number of sensors or the like configured to sense and/or collect bio-signals such as cardiac electrical signals radiating from the heart and/or mechanical or other signals (e.g., one or more pressure signals) in the mediastinum.

The first sensor 244a and the second sensor 244b are configured to measure different health characteristics associated with the heart of the patient P. In some embodiments, the first sensor 244a and the second sensor 244b can measure continuously, periodically, or when a command is received by the first sensor 244a and the second sensor 244b (e.g., received from the processor 222). In some embodiments, the first sensor 244a is configured to detect and/or sense cardiac signals. In some embodiments, the first sensor 244a is configured to detect and/or sense cardiac electrical signals (e.g., heart rate, voltage, P wave, QRS morphology, ST segment, T wave, ECG signals, etc.) that radiate from the heart. In some embodiments, the first sensor 244a can be positioned so that it engages the heart H of the patient P and/or is disposed in or on the body of the patient P such that the cardiac signals can be measured (e.g., in contact with or in close proximity to the pericardium).

In some embodiments, the second sensor 244b is a different sensor than the first sensor 244a. In some embodiments, the second sensor 244b is configured to detect and/or sense mechanical signals. In some embodiments, the second sensor 244b is configured to detect and/or sense hemodynamic status. In some embodiments, the second sensor 244b is a pressure sensor. For example, the second sensor 244b may be a pressure sensor configured to detect and/or sense hemodynamic pressure. In some embodiments, the second sensor 244b is a pressure sensor configured to detect and/or sense pressure or changes in pressure in the substernal space and/or anterior mediastinum associated with cardiac movement, respiration, and/or the like. In some embodiments, the second sensor 244b is a pressure sensor in a hermetically sealed housing. In some embodiments, the second sensor 244b can be positioned so that it engages the heart H (e.g., the pericardium) of the patient P or can be positioned in any other place in or on the body of the patient P where the second sensor 244b can detect and/or sense a hemodynamic and/or mechanical status of the heart. In some embodiments, the second sensor 244b is in the anterior mediastinum to detect and/or sense pressure changes that can be indicative of a hemodynamic status (and/or a respiratory status or other status, which in some instances, can be used to determine and/or confirm hemodynamic status). For example, the second sensor 244b can be configured to detect and/or sense mechanical signals such as changes in pressure in the anterior mediastinum as a result of the beating of the heart H or respiration (inflation/deflation of the lungs). Alternatively, the mechanical signals can be associated with characteristics such as position, acceleration, and/or the like.

In some embodiments, the first sensor 244a and/or the second sensor 244b is/are located in the epicardium of the heart. In some embodiments, the first sensor 244a and/or the second sensor 244b is/are located within the heart. For example, the first sensor 244a and/or the second sensor 244b may be located in the right heart. In some embodiments, the first sensor 244a and the second sensor 244b can be collocated or substantially collocated. In some embodiments, the first sensor 244a and/or the second sensor 244b can be remotely or separately located. For example, in some embodiments, the first sensor 244a can be positioned in contact with or adjacent to the fibrous pericardium of the heart H, while the second sensor 244b can be positioned apart from the heart H in the substernal space. In some embodiments, the first sensor 244a and/or the second sensor 244b detects and/or senses characteristics of the heart H, lungs, and/or other portions or body parts of the patient P.

As described in further detail herein, the sensors 244a and 244b can measure, detect, and/or sense one or more characteristics associated with the functioning of the heart H, the respiratory system, and/or other portions or body parts of the patient P and can send data associated with those characteristics to the generator 220 (e.g., directly or indirectly via the lead 240). Upon receipt, the generator 220 can analyze, process, aggregate, correlate, verify, confirm, corroborate, etc. the data to determine, for example, a cardiac status of the heart H. Furthermore, based on the determined, defined, and/or confirmed cardiac status, the generator 220 can detect and/or determine the occurrence of health events, such as arrhythmia, tachycardia, and/or the like. In some implementations, the use of data from each of the first sensor 244a and the second sensor 244b can allow the generator 220 to determine the cardiac status of the heart H with greater sensitivity and specificity than when determining cardiac status using cardiac signals, QRS complex morphology, and/or other cardiac electrical signal measurements alone (e.g., determined based on signals from the first sensor 244a), hemodynamic status or mechanical signal measurements alone (e.g., determined based on signal from the second sensor 244b), and/or other characteristics individually.

In some implementations, the process of the generator 220 determining the cardiac status and/or functioning of the heart can include, for example, comparing, correlating, verifying, confirming, corroborating, and/or synchronizing the signal data received from the first sensor 244a and the second sensor 244b. For example, the first sensor 244a can be configured to determine and/or sense cardiac electrical signals and the second sensor 244b can be configured to determine and/or sense mechanical signals such as, for example, pressure changes and/or the like (e.g., in the anterior mediastinum) that can be indicative of hemodynamic status and/or output of the heart H. The generator 220 can receive the signal data from the sensors 244a and 244b and can determine the cardiac status and further classify cardiac arrhythmia, based at least in part on comparing, correlating, verifying, confirming, corroborating, and/or synchronizing a cardiac electrical signal curve (from the first sensor 244a) and a mechanical signal curve (from the second sensor 244b). Thus, determining a cardiac status and a classification of cardiac arrhythmia can include the generator 220 determining whether a sensed or determined cardiac rhythm (e.g., based on cardiac electrical signal data received from the first sensor 244a) has an expected, verified, and/or confirmed corresponding sensed or determined hemodynamic status and/or output (e.g., based on mechanical signal data received from the second sensor 244b such as pressure changes in the anterior mediastinum resulting from the mechanical functioning (pumping) of the heart, respiration, and/or the like).

The generator 220 and/or the processor 222 thereof can be configured to use the data associated with the electrical signals (e.g., radiating from the heart H) to determine and/or define a suspected or assumed cardiac status. In addition, the processor 222 can be configured to confirm and/or verify the suspected and/or assumed cardiac status based at least in part on a correlation, corroboration, etc. between the electrical signal data (received from the first sensor 244a) and the mechanical (or other) signal data (received from the second sensor 244b). For example, a relatively high degree of correlation between the electrical signal data and the mechanical signal data can be confirmation of the suspected and/or assumed cardiac status because the mechanical functioning of the heart H corresponds to and/or is correlated with the electrical functioning of the heart H. Conversely, the processor 222 can be configured to determine that the suspected and/or assumed cardiac status based on the electrical signal data alone is not the actual cardiac status when there is a relatively low degree of correlation between the electrical signal data and the mechanical signal data. In some implementations, correlating data associated with two different signals and/or otherwise using additional data to confirm and/or verify an implied, suspected, and/or assumed cardiac state (e.g., based on a single data source like cardiac electrical signals) can reduce and/or limit first positives, which can lead to inappropriate or undesired shock treatments delivered to the heart H of the patient P. Moreover, if a hemodynamic status and/or output is not what is expected, based on the determined cardiac rhythm), the generator 220 may withhold, delay, and/or modify the therapy. In some instances, if the characteristics associated with and/or indicative of hemodynamic status are not as expected, the generator 220 can modify or augment the predicted status of the heart H.

In some embodiments, one or more machine learning algorithms may be employed to define, determine, correlate, verify, confirm, corroborate, etc. expected hemodynamic status(es) and/or output(s) for a given cardiac rhythm. In some embodiments, one or more machine learning algorithms may be employed to define, determine, correlate, verify, confirm, and/or predict a cardiac status based at least in part on one or more relationships and/or a degree of correlation between, for example, data received from the first sensor 244a (e.g., cardiac electrical signal data such as, for example, cardiac rhythm) and data received from the second sensor 244b (e.g., mechanical (or other) signal data such as, for example, hemodynamic status and/or output).

FIG. 5 schematically depicts a diagnostic/treatment system 300 engaging with a patient P according to another embodiment. The diagnostic/treatment system 300 is structurally and/or functionally similar to the diagnostic/treatment system 100 and/or 200 of FIGS. 3 and/or 4, respectively. The diagnostic/treatment system 300 includes a cardiac treatment device 310 (e.g., functionally and/or structurally similar to the cardiac treatment device 110 of FIG. 1) engaging with the heart H of the patient P. The cardiac treatment device 310 includes a generator 320 (e.g., structurally and/or functionally similar to the generator 120 and/or 220 of FIGS. 3 and/or 4, respectively) operatively coupled to a lead 340 (e.g., structurally and/or functionally similar to the lead 240 of FIG. 4). The lead 340 includes a sensor interface 342 (e.g., structurally and/or functionally similar to the sensor interface 242 of FIG. 4).

The cardiac treatment device 310 includes a first sensor 344a (e.g., structurally and/or functionally similar to the first sensor 244a) and a second sensor 344b (e.g., structurally and/or functionally similar to the second sensor 244b). In the embodiment shown in FIG. 5, the first sensor 344a and the second sensor 344b are integrated into the lead 340. In some embodiments, such as depicted in FIG. 4, at least one of the first sensor 344a and/or the second sensor 344b are remote from the lead. The sensor interface 342 may be configured to physically couple the first sensor 344a and the second sensor 344b to the lead 340. The sensor interface 342 may be configured to receive and send the outputs from the first sensor 344a and the second sensor 344b. In some embodiments, the sensor interface 342 may preprocess and/or synchronize the signals from the first sensor 344a and the second sensor 344b.

In some implementations, integrating the first sensor 344a and the second sensor 344b can allow for the sensors 344a and/or 344b to be positioned close to the heart H of the patient P. The first sensor 344a may be positioned along the lead 340 such that cardiac signals can be measured. The second sensor 344b may be positioned along the lead 340 such that hemodynamic status or output can be measured. In some embodiments, the first sensor 344a and/or the second sensor 344b are positioned along a region, portion, or section of the lead 340. For example, the first sensor 344a and/or the second sensor 344b may be positioned along a distal section corresponding to a portion of the lead 340 nearest or proximate (e.g., within 1%, within 2%, within 3%, within 4%, within 5%, within 10%, within 25%, etc.) the distal end of the lead 340. In some embodiments, the first sensor 344a and/or the second sensor 344b is/are positioned at the distal end of the lead 340. In some implementations, integrating the first sensor 344a and the second sensor 344b directly into the lead 340 can potentially decrease the noise in the signals from the first sensor 344a and the second sensor 344b as the signal may have to travel a shorter distance and/or through fewer interfaces.

While the diagnostic/treatment system 300 is described above as including “the first sensor 344a” and “the second sensor 344b,” it should be understood that the first sensor 344a can be a single sensing device or multiple sensing devices that collectively function as the first sensor 344a, and similarly, the second sensor 344b can be a single sensing device or multiple sensing devices that collectively function as the second sensor 344b. In some implementations, multiple sensing devices can allow for sensor data that includes multiple signal vectors (e.g., multiple cardiac electrical and/or mechanical signal vectors). Similarly, while the diagnostic/treatment system 300 is described herein as including “the first sensor 344a” and “the second sensor 344b,” it should be understood that the diagnostic/treatment system 300 can include any number of additional sensors configured to sense and/or detect any suitable characteristic(s) associated with the patient (e.g., cardiac electrical and/or mechanical signals or any suitable non-cardiac signals). In some implementations, each sensor can be configured to sense and/or detect a different characteristic associated with the heart, or more generally, the patient. In some implementations, one or more sensors can be configured to sense or detect the same characteristic, thereby allowing for confirmation/verification of signal data and/or a desired degree of sensitivity and/or specificity in interpreting the signal data.

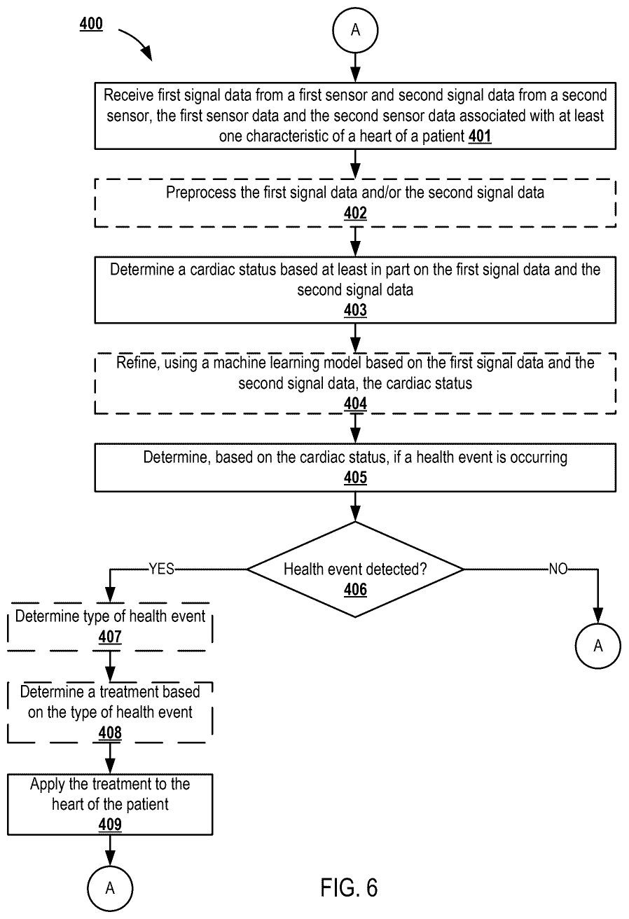

FIG. 6 is a flow chart depicting a method 400 for determining if a patient is having a health event and whether to apply a corresponding corrective action according to an embodiment. In some embodiments, the method 400 can be executed and/or performed by a diagnostic/treatment system that is functionally and/or structurally similar to the diagnostic/treatment system 200 of FIG. 4 and/or the diagnostic/treatment system 3 of FIG. 5. In some embodiments, the method 400 can be executed by one or more devices. For example, the method 400 can be executed by the diagnostic/treatment system 300 of FIG. 5. The method 400 is configured to use data associated with at least two characteristics of the heart or a function of the heart. In some implementations, the method 400 is configured to use at least two signals from at least two data sources (e.g., sensors) to determine if a patient is having a health event (e.g., cardiac arrest, tachycardia, arrhythmias). Using at least two signals reduces the likelihood that the diagnostic/treatment system determines a health event is occurring when compared to a diagnostic/treatment system that uses only one signal for determining if a health event is occurring. Additionally, the method 400 allows for determining the type of corrective action (e.g., treatment) to be determined based on the health event determined. This allows the diagnostic/treatment system to more accurately treat (e.g., with greater sensitivity and specificity) the health event and reduce the likelihood of sudden cardiac arrest. The method 400 may improve sensitivity and specificity of treatment for ventricular tachycardia or ventricular fibrillation.

The method 400 includes, at 401, receiving signal data from a first sensor (e.g., functionally and/or structurally similar to the first sensor 244a of FIG. 4 and/or the first sensor 344a of FIG. 5) and second signal data from a second sensor (e.g., structurally and/or functionally similar to the second sensor 244b of FIG. 4 and/or the second sensor 344b of FIG. 5). The first signal data and the second signal data are associated with at least one characteristic of a heart of a patient. In some embodiments, the first sensor is a sensor configured to measure or sense cardiac electrical signals and the first signal data is associated with, for example, cardiac signals, QRS complex morphology, and/or the like. In some embodiments, the second sensor is a sensor configured to measure mechanical signals (e.g., hemodynamic status, pressure changes, and/or the like) and the second signal data is associated with mechanical signals (e.g., hemodynamic status data or a proxy or derivative thereof such as changes in pressure in the anterior mediastinum) and/or output of the heart of the patient. In some embodiments, the at least one characteristic can include a first characteristic and a second characteristic. The first characteristic can be associated with a status (e.g., physiological status(es), pathophysiological status(es), and/or the like, or combinations thereof) and the second characteristic can be associated with the same or different status. For example, the at least two characteristics can include an electrical characteristic associated with a status and a mechanical characteristic associated with the status. As another examples, the at least two characteristics can include a characteristic (electrical, mechanical, etc.) associated with a first status and a characteristic (electrical, mechanical, etc.) associated with a second status different from the first status. In some embodiments, 401 may include receiving additional signal data from any number of additional sensors and/or additional data sources (e.g., one or more ex vivo data sources). In some embodiments, 401 may include receiving at least one of the first signal data and/or the second signal data from an external data source (e.g., patient database, etc.).

At 402, the method 400 optionally includes preprocessing the first signal data and/or the second signal data. Preprocessing can include noise reduction, filtering, amplification, normalization, conversion (e.g., analog-to-digital, etc.), synchronization, and/or the like. Preprocessing can prepare for the signals to be received by another component of the diagnostic/treatment system. For example, a sensor interface (e.g., functionally and/or structurally similar to the sensor interface 242 of FIG. 4 and/or the sensor interface 342 of FIG. 5) can preprocess the first signal data and the second signal data so that the first signal data and the second signal data can be received by a generator (e.g., a treatment generator such as an ICD generator structurally and/or functionally similar to the generator 220 of FIG. 4 and/or the generator 320 of FIG. 5) and/or a compute device. In some embodiments, the compute device and/or the generator may preprocess the first signal data and/or the second signal data to prepare the data for processing.

At 403, the method 400 includes determining a cardiac status based at least in part on the first signal data and the second data signal. The cardiac status can correspond to the status of the heart (e.g., heartbeat profile, cardiac activity, etc.). In some embodiments, determining a cardiac status can further be based on data from additional source(s) (e.g., additional sensors and/or one more ex vivo data sources). Determining cardiac status can include, for example, comparing and synchronizing the signal data and artifacts in the signal data to determine the cardiac status or functioning of the heart. For example, when the first signal data is associated with cardiac electrical signals (e.g., cardiac signals or used to determine cardiac rate) and the second signal data is associated with hemodynamic status and/or output, determining cardiac status can include synchronizing an electrical signal curve (from the first signal data) and a pressure curve (from the second signal data) to correlate the signal data. As another example, determining the cardiac status can include determining if a rhythm seen in the cardiac electrical signal data has an expected corresponding result in the hemodynamic status and/or output signal data. As such, using the first signal data and the second signal data provides a more sensitivity and specificity in determining cardiac status than when determining cardiac status using just one source of data (e.g., cardiac electrical signals alone).

At 404, the method 400 optionally includes refining the cardiac status using a machine learning model based on the first signal data and the second signal data. Refining can include, for example, improving, tuning, updating, and/or verifying the cardiac status determined at 403. The machine learning model can be trained based on cardiac status associated with data form the first sensor and the second sensor. For example, the machine learning model can be trained based on cardiac status associated with cardiac electrical signal data (e.g., cardia rate) and hemodynamic status and/or output data from the patient, historical data, data from studies, medical professional input, and/or the like. In some embodiments, the machine learning model can be trained based on additional patient data (e.g., activity level, height, weight, age, and/or the like). Training on patient data allows for the machine learning model to gain further insight on the patient that may affect the characteristics of the heart measured by the sensors.

In some embodiments, the cardiac status may be changed, updated, tuned, etc., based at least in part on comparing the cardiac status from 403 to more closely align with empirical data, a status determination by one or more trained cardiologists, updated clinical evidence, and/or any suitable known or accepted ground truth. In some embodiments, the machine learning model may be configured to recognize long-term changes in cardiac status and/or the first signal data and/or the second signal data and adapt to these changes. For example, the machine learning model may recognize that hemodynamic output has been decreasing over weeks, months, or years. Recognizing changes allows the machine learning model to adapt the treatment (e.g., ICD shock therapy, cardiac pacing, etc.) along with the patient as the patient changes. In some embodiments, the machine learning model may receive feedback data and/or training sets that are used to further train the machine learning model.

At 405, the method 400 includes determining, based on the cardiac status, if a health event is occurring. Determining if a health event is occurring can include, in some embodiments, comparing the cardiac status to an expected cardiac status. For example, if the cardiac status indicates that a heartbeat is higher than it expected, the presence of a health event may be determined. In some embodiments, a machine learning model can be used to determine if a health event is present based at least in part on the cardiac status. For example, the machine learning model can recognize changes in the patient's heart activity that can indicate the presence of a health event, or if no health event is occurring. In some embodiments, an indication that a health event is occurring may be sent to the user. In some embodiments, the machine learning model can recognize when the patient's heart activity is expected and/or predicted to increase. For example, when the patient is exercising, experiencing stress or anxiety, and/or the like.

At 406, the method 400 includes a decision of whether a health event has been detected. If a health event has not been detected, the method 400 returns to 401 to continue monitoring the heart of the patient. If a health event has been detected, the method 400 continues to 407.

At 407, the method 400 optionally includes determining a type of health event. In some embodiments, determining a type of health event can include comparing and matching the cardiac status to known health events. In some embodiments, a machine learning model may be used to match the cardiac status to a health event. In some embodiments, the type of health event can be sent to a user device and/or the like for review by the user. For example, the type of health event can be reviewed by the patient, a medical proxy, a medical professional, and/or the like. At 408, the method 400 optionally includes determining a treatment based on the type of health event determined in 407. The treatment can correspond to the type of health event and/or can be adjusted based on information about the patient and/or the characteristics of the heart (e.g., heartrate, blood pressure, arrhythmia classification, cardiac status, hemodynamic status, etc.) at the time of or leading up to the health event. Determining the treatment based on the type of health event allows for a more accurate treatment for the given health event than when a predetermined treatment is used for any/all detected health events. For example, if, at 407, the health event is determined and/or predicted to be ventricular tachycardia, the treatment may be anti-tachycardia pacing. On the other hand, if the health event is determined and/or predicted to be ventricular fibrillation, the treatment may be shock therapy (e.g., defibrillation shock(s) generated by an ICD generator). In some embodiments, 408 may include determining if the health event is still occurring. If the health event is determined to no longer be occurring, 408 may include determining that a treatment is not desired. Said another way, step 408 can include delaying potential treatment to confirm that the health event is still occurring, which in some instances, may reduce what may otherwise be an “appropriate” shock therapy.

At 409, the method 400 includes applying the treatment to the heart of the patient. The treatment, either predetermined or as determined in 408, is generated by the generator and delivered to the heart via the lead. After the treatment is delivered to the heart, the method 400 returns to 401 to continue monitoring the cardiac activity of the heart of the patient. The method 400 repeats as long as the cardiac treatment device (e.g., ICD) is powered and implanted in the patient.

While the method 400 is described above as receiving first signal data from the first sensor and second signal data from the second sensor, it should be understood that the first sensor can be a single sensing device or multiple sensing devices that collectively function as the first sensor, and similarly, the second sensor can be a single sensing device or multiple sensing devices that collectively function as the second sensor. In some implementations, multiple sensing devices can allow for sensor data that includes multiple signal vectors (e.g., multiple cardiac electrical and/or mechanical signal vectors).

In some implementations, the method 400 may optionally include receiving signal data from one or more additional sensors (e.g., in addition to the first sensor and the second sensor). In some implementations, each sensor can be configured to sense and/or detect a different characteristic associated with the heart, or more generally, the patient. In some implementations, one or more sensors can be configured to sense or detect the same characteristic, thereby allowing for confirmation/verification of signal data and/or a desired degree of sensitivity and/or specificity in interpreting the signal data. For example, in some implementations, the step at 401 can include receiving signal data from any number of sensors or data sources included in or in communication with the cardiac treatment device (e.g., ICD); the optional step at 402 may include preprocessing the signal data from each of the sensors; the step at 403 may include determining the cardiac status based at least in part on each or all of the signal data; and the optional step at 404 may include refining the cardiac status using a machine learning model based on each or all of the signal data.

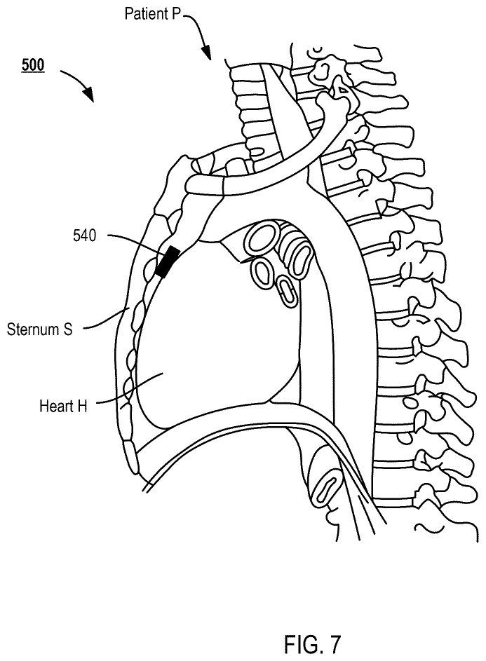

FIG. 7 depicts a placement of a lead 540 in the chest of a patient according to an embodiment. In some embodiments, the lead 540 is structurally and/or functionally similar the lead 240 of FIG. 4 and/or the lead 340 of FIG. 5. The lead 540 is coupled to a generator (e.g., a generator structurally and/or functionally similar to the generator 220 of FIG. 4 and/or the generator 320 of FIG. 5) to form a cardiac treatment device such as an ICD (and/or treatment device structurally and/or functionally similar to the cardiac treatment device 210 of FIG. 4 and/or the cardiac treatment device 310 of FIG. 5). The position of the lead 540 can be a parameter and/or factor in reducing anti-tachycardia pacing (ATP) thresholds. ATP thresholds that are higher than desired could result in inconsistent pacing, intolerable sensations to the patient during pacing, or non-use of pacing that increases probability of undesirable health events and/or undesirable shocks.

As seen in FIG. 7, the lead 540 is positioned in the substernal space between a sternum S and a pericardium of a heart H of a patient P. The lead 540 is positioned in the substernal space since any space between the electrode and the pericardium can increase pacing thresholds. Furthermore, the lead 540 is configured to facilitate the natural movement of the heart H that occurs with each cardiac cycle. In some embodiments, the lead 540 can facilitate natural movement by using the sternum S as a base and having the lead 540 pressed against or otherwise placed in contact with the fibrous layer of the pericardium. This position allows for the lead 540 to move in multiple directions and absorb and/or move with the motion of the heart H.

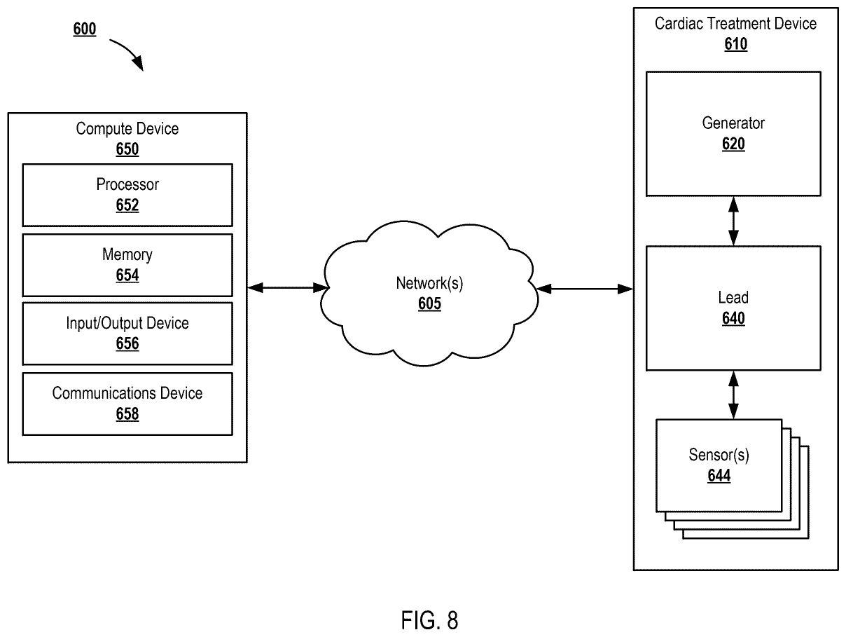

FIG. 8 depicts a cardiac treatment device 610 (e.g., functionally and/or structurally similar to the cardiac treatment device 210 of FIG. 4 and/or the cardiac treatment device 310 of FIG. 5) in communication with an external compute device 650 via network(s) 605 according to an embodiment. In some embodiments, the cardiac treatment device 610 can be an ICD, CRT-D, and/or any other implantable cardiac treatment device. The network(s) 605 can include one or more network(s) that may be any type of network (e.g., a local area network (LAN), a wide area network (WAN), a virtual network, a telecommunications network) implemented as a wired network and/or wireless network (e.g., Wi-Fi, Bluetooth®, Bluetooth® low energy, Zigbee, Z-Wave, Thread, Matter, etc.) and used to operatively couple to any compute device, including, for example, the compute device 650 and/or the cardiac treatment device 610. In some embodiments, the components of the cardiac treatment device 610 can be configured to communicate directly with the network(s) 605. For example, the generator 620 (e.g., structurally and/or functionally similar to the generator 220 of FIG. 4 and/or the generator 320 of FIG. 5) may send and receive information associated with cardiac status, health events, and/or treatment to the compute device 650 (or any other suitable device) via the network(s) 605. As another example, the lead 640 (e.g., structurally and/or functionally similar to the lead 240 of FIG. 4 and/or the lead 340 of FIG. 5) may send and/or receive processed signal data to compute device 650 (or any other suitable device) via the network(s) 605. As another example, the sensor(s) 644 (e.g., structurally and/or functionally similar to the sensor(s) 144 of FIG. 3, the first sensor 244a and/or the second sensor 244b of FIG. 4, and/or the first sensor 344a and/or the second sensor 344b of FIG. 5) can send and receive information associated with the signals and/or the operation of the sensor(s) 644.

The compute device 650 is an external device configured to receive and send signals, data, and/or information from/to the cardiac treatment device 610 via the network(s) 605. The compute device 650 may be any suitable device or combination of devices. For example, the compute device 650 can be any suitable electronic device configured to send, receive, process, analyze, store, use, change, define, etc. data, data structures, and/or the like. The components of the compute device(s) can be contained within a single housing or machine or can be distributed within and/or between multiple physical machines, virtual machines, and/or any combination thereof. In some embodiments, the compute device(s) 650 can be physically included in and/or on local machine(s) or device(s) or can be stored, run, executed, and/or otherwise implemented in and/or on remote machine(s) or device(s). For example, the compute device 650 (or a portion or component thereof) can be and/or can include, but is not limited to, personal computer(s) (PC), laptop PC(s), tablet PC(s), mobile device(s) (e.g., a smart phone, wearable, etc.), server device(s), workstation(s), and/or the like. In some embodiments, the compute device 650 or at least a portion thereof can be implemented as a virtual machine and/or virtual private server executed on and/or run as an instance or guest on a physical machine and/or cloud platform like Microsoft Azure®, Amazon® web services, IBM® cloud computing, etc.

The compute device 650 includes a processor 652 a memory 654, an input/output (I/O) device 656, and a communication device 658. The processor 652 can be and/or can include one or more data processors, image processors, graphics processing units (GPU), digital signal processors (DSP), analog signal processors, mixed-signal processors, machine learning processors, deep learning processors, finite state machines (FSM), compression processors (e.g., data compression to reduce data rate, bandwidth, and/or memory requirements), encryption processors (e.g., for secure wireless data and/or power transfer), and/or the like. The processor 652 can be, for example, a general-purpose processor, central processing unit (CPU), microprocessor, microcontroller, Field Programmable Gate Array (FPGA), an Application Specific Integrated Circuit (ASIC), a processor board, a virtual processor, and/or the like. The underlying device technologies may be provided in a variety of component types such as metal-oxide semiconductor field-effect transistor (MOSFET) technologies like complementary metal-oxide semiconductor (CMOS), bipolar technologies like generative adversarial network (GAN), polymer technologies (e.g., silicon-conjugated polymer and metal-conjugated polymer-metal structures), mixed analog and digital technologies, and/or the like.

The memory 654 can be, for example, a random-access memory (RAM), static RAM (SRAM), dynamic RAM (DRAM), a synchronous dynamic RAM (SDRAM), a memory buffer, a read-only memory (ROM), an erasable programmable read-only memory (EPROM), an electrically erasable read-only memory (EEPROM), flash memory, volatile memory, non-volatile memory, combination(s) thereof, types or subtypes or variations thereof, and/or the like. In some embodiments, the memory 654 can store instructions to cause the processor to execute modules, processes, and/or functions associated with the compute device, such as sending/receiving data, aggregating data, analyzing data, executing any number of models or algorithms (e.g., machine learning models, etc.), controlling any number of controllers, machines, devices, and/or components.

The input/output (I/O) device 656 can be and/or can include any suitable device(s), interface(s), port(s), etc. that can allow the compute device 650 to receive an input and/or to provide an output. For example, in some implementations, an input can include a port or wireless communication device configured to communicate with and/or receive input from a keyboard, mouse, and/or any other peripheral device. In some implementations, an output can include a port or wireless communication device configured to communication with and/or provide output to an audio device, a display device, a haptic device, and/or any other suitable device. For example, an I/O device 656 can be, can include, and/or can be configured to at least partially control a display that can provide at least a portion of a user interface for a software application (e.g., a mobile application, a PC application, an internet web browser, etc.) installed and/or executed on or by the compute device (or the processor thereof). In such implementations, the display can be, for example, a cathode ray tube (CRT) monitor, a liquid crystal display (LCD) monitor, a light emitting diode (LED) monitor, and/or the like.

The communication device 658 can be any suitable device(s) and/or interface(s) that can communicate with and/or via the network(s) 605. The communication device 658 can include one or more wired and/or wireless interfaces, such as, for example, Ethernet interfaces, optical carrier (OC) interfaces, and/or asynchronous transfer mode (ATM) interfaces. In some embodiments, the communication device 658 can be, for example, a network interface card and/or the like that can include at least an Ethernet port and/or a wireless radio (e.g., a Wi-Fi® radio, a Bluetooth® radio, near field communications (NFC) radio, etc.). In some embodiments, the communication device 658 can be configured to receive/send signals and/or data from/to any number of devices, data sources, assets, machines, controllers, sensors, systems, etc. via the network. Moreover, the communication device 658 is in communication with the processor 652, the memory 654, and the I/O device 656 allowing signals and/or data to be transmitted therebetween.

In some embodiments, the compute device 650 may be configured to execute any of the processes and/or any suitable portion of the processes described in reference to the cardiac treatment devices described herein, such as the cardiac treatment device 210 of FIG. 4 and/or the cardiac treatment device 310 of FIG. 5. For example, the compute device 650 may be configured to store instructions for and/or execute instructions related to using machine learning models and/or algorithms. The compute device 650 can be configured to complete computations that may be too resource (e.g., battery power, memory, processing power, etc.) for the cardiac treatment device 610.

For example, the compute device 650 may be configured to execute, via the processor 652, machine learning model processes for the cardiac treatment device 610. In some embodiments, the compute device 650 may be configured to receive signals and/or data associated with the sensor(s) 644, determine a cardiac status and a health event occurring, and/or determine a treatment for the health event. In some embodiments, the compute device 650 may be configured to send commands and/or outputs to the cardiac treatment device 610 so that the cardiac treatment device 610 can, in turn, deliver treatment to the heart of the patient. In some embodiments, the compute device 650 may be configured to send commands and/or outputs to the cardiac treatment device 610 that can cause the cardiac treatment device 610 to update firmware, data stored in one or more repositories (e.g., databases), and/or instructions stored in the memory 654. As such, the cardiac treatment device 610 can adapt to treatment decisions based on patient changes over time.

The input/output device 656 of the compute device can include a device to input signals and/or commands into the compute device 650 and/or output signals and/or commands. For example, the input/output device 656 can include a device (e.g., touchscreen, keyboard, mouse, etc.) for inputting inputs by a user (e.g., doctor, patient, caretaker, etc.) to input information associated with the patient and regarding the operation of the cardiac treatment device 610. In some embodiments, a user may input training sets for a machine learning model. The I/O device 656 may include a device (e.g., screen, light, etc.) configured to output information regarding the cardiac treatment device 610. For example, the I/O device 656 can include a display and/or the like configured to generate a graphical and/or visual output representing the status (e.g., battery status, operational status, etc.) of the cardiac treatment device 610, information associated with health events, information associated with treatment, and/or the like.

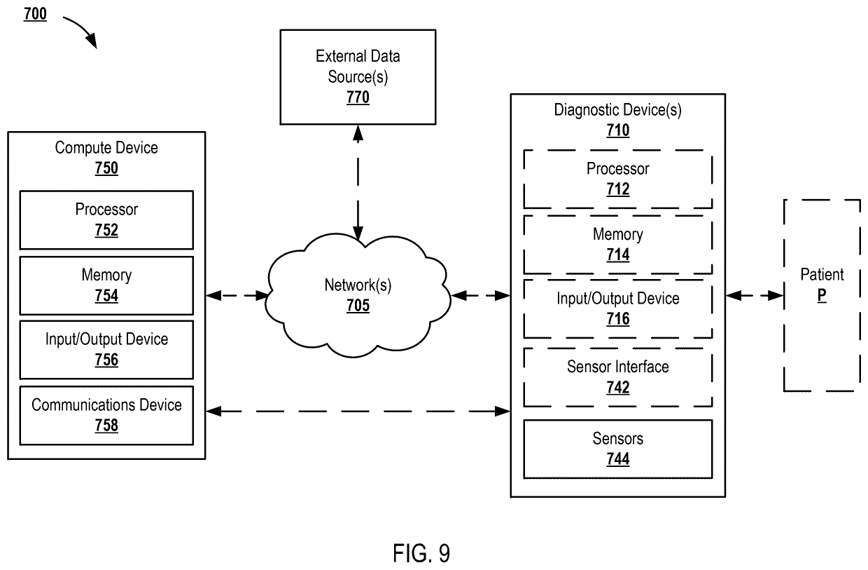

FIG. 9 schematically depicts a diagnostic/treatment system 700 engaging a patient P, according to an embodiment. In some embodiments, the diagnostic/treatment system 700 can be similar to and/or can be a specific implementation of the diagnostic/treatment system 100 described above with reference to FIG. 3. As shown, the diagnostic/treatment system 700 (“system 700”) includes one or more diagnostic device(s) 710 that can be utilized for monitoring a patient, determining if a health event is occurring, and/or diagnosing a patient. As described herein, the diagnostic/treatment system 700 can be used to make diagnostic predictions and/or the like based at least in part on data from multiple data sources, which in turn, can decrease the likelihood of a false positive diagnosis and/or delivery or an undesired or inappropriate therapy or treatment. Although the diagnostic/treatment system 700 is described herein with reference to FIG. 9 as being used to monitor and/or diagnose (or make diagnostic predictions based on data associated with) a patient, in some embodiments, the system 700 (and/or the diagnostic device(s) 710 or any other device(s) included in the system 700) can be used to make therapeutic and/or treatment decisions and/or otherwise deliver one or more therapies or treatments that may, for example, correspond to and/or treat a disease state or health condition that is being monitored by or that is diagnosed and/or predicted by the diagnostic device(s) 710. In this manner, the diagnostic device 710 can be similar to, can include, and/or can otherwise perform functions associated with the cardiac treatment devices 210, 310, and/or 610.

The diagnostic/treatment system 700 is shown in FIG. 9 with the diagnostic device(s) 710 engaging a patient P (e.g., implanted or at least partially implanted in a patient P). The diagnostic/treatment system 700 also includes a compute device 750 and external data source(s) 770 that may be communicably coupled to the diagnostic device 710 via one or more network(s) 705. Any of the components, devices, and/or aspects of the system 700 can be similar in at least form and/or function to corresponding components, devices, and/or aspects of the system 600 described above with reference to FIG. 8. Accordingly, some such components, devices, and/or aspects of the system 700 may not be described in further detail herein and should be considered as structurally and/or functionally similar to the corresponding components, devices, and/or aspects of the system 600 unless stated otherwise.

The network(s) 705 shown in FIG. 9 can be and/or can include one or more network(s) that may be any type of network or combination of networks (e.g., LANs, WANs, virtual networks, telecommunication networks, etc.) implemented as a wired network and/or wireless network (e.g., Wi-Fi, Bluetooth®, Bluetooth® low energy, Zigbee, Z-Wave, NFC, Thread, Matter, etc.). Accordingly, the network(s) 705 can be used to operatively couple any number of compute devices (or other electric or electronic devices) including, for example, the compute device 750, the external data source(s) 770, and/or the diagnostic device(s) 710.

The compute device 750 shown in FIG. 9 is and/or includes one or more external devices (e.g., external to the diagnostic device 710 and/or the patient P) that is/are in communication with the external data source(s) 770 and the diagnostic device(s) 710 via the network(s) 705. The compute device 750 may be any suitable device or combination of devices configured to send, receive, process, analyze, store, use, change, define, etc. data, data structures, and/or the like. Moreover, the compute device 750 can be configured to perform one or more processes, functions, applications, programs, algorithms, models, etc. The components of the compute device(s) 750 can be contained within a single housing or machine or can be distributed within and/or between multiple physical machines, virtual machines, and/or any combination thereof. In some embodiments, the compute device 750 can be physically included in and/or on local machine(s) or device(s) or can be stored, run, executed, and/or otherwise implemented in and/or on remote machine(s) or device(s). For example, the compute device 750 (or a portion or component thereof) can be and/or can include, but is not limited to, PC(s), laptop(s), tablet(s), mobile device(s) (e.g., a smart phone, wearable, etc.), server(s), workstation(s), and/or the like. In some embodiments, the compute device 750 or at least a portion thereof can be implemented as a virtual machine and/or virtual private server executed on and/or run as an instance or guest on a physical machine and/or cloud platform like Microsoft Azure®, Amazon® web services, IBM® cloud computing, etc. In some embodiments, the compute device 750 may be associated with a healthcare service provider such as a doctor, hospital, or medical center, and/or a user such as an emergency responder, a healthcare professional, a patient, and/or the like.

The compute device 750 includes a processor 752, a memory 754, an input/output device 756, and a communications device 758. The compute device 750 can be functionally and/or structurally similar to or substantially the same as the compute device 650. For example, the processor 752 can be and/or can include one or more data processing units, engines, modules, devices, circuits, controllers, etc. such as any of those described above with reference to the processor 652. The memory 754 can be and/or can include one or more volatile or non-volatile memories or data storage devices or structures such as any of those described above with reference to the memory 654. The input/output (I/O) device 756 can be and/or can include any suitable device(s), interface(s), port(s), etc. that can allow the compute device 750 to receive an input and/or to provide an output, as described above with reference to the I/O device 656. The communication device 758 can be any suitable device(s) and/or interface(s) that can communicate with and/or via the network(s) 705, as described above with reference to the communication device 658. Moreover, the communication device 758 is in communication with the processor 752, the memory 754, and the I/O device 756 allowing signals and/or data to be transmitted therebetween (e.g., via a system bus and/or the like).

The external data source(s) 770 shown in FIG. 9 can be one source or multiple sources storing and/or otherwise configured to store information. The external data source(s) 770 can be and/or can include one or more repositories, databases, servers, and/or other data sources that is/are in communication with the compute device 750 and the diagnostic device(s) 710 via the network(s) 705. In some implementations, the external data source(s) 770 store and/or are configured to store information associated with the patient P, health-related data, machine learning and/or AI training data sets, and/or the like. For example, the external data source(s) 770 can include and/or can store data associated with training data sets configured to train any of the machine learning models described herein, information associated with the patient P (e.g., demographics information, age, sex, weight, height, health history, diagnoses, health trends, health predictions, treatment programs or modalities, sensitivities, allergies, etc.), and/or the like. In some embodiments, the external data source(s) 770 can include and/or can store data associated with any number of patients and/or any suitable non-patient-specific data such as environmental data (e.g., that may impact health), community or other broader-population health-related data, and/or any other suitable data. Moreover, the external data source(s) 770 can include and/or can store relationships, associations, correlations, etc. associated with such data patient-specific or non-patient-specific data.

In some implementations, the external data source(s) 770 can be configured to send, via the network(s) 705, patient-specific data, non-patient-specific data, and/or any other suitable data to at least the compute device 750, which in turn, can process, analyze, and/or otherwise use the data to, for example, define, update, train, and/or execute one or more machine learning algorithms or models associated with defining and/or generating one or more diagnoses or diagnostic predictions (e.g., specific to the patient P or associated with a broader population). In addition, the external data source(s) 770 can be configured to receive, via the network(s) 705, patient-specific data, non-patient specific data, and/or any other suitable data from the compute device 750 and/or the diagnostic device(s) 710. In response, the external data source(s) 770 can store the data and/or otherwise update data already stored.

The diagnostic device(s) 710 shown in FIG. 9 can include one or more device(s) configured to sense, detect, monitor, and/or measure bio-signals associated with one or more characteristics (e.g., physiological characteristics, pathophysiological characteristics, and/or the like, or combinations thereof) inside or outside of the body of the patient P. The diagnostic device(s) 710 can be implanted in the body of the patient P or can be disposed outside of the patient P. For example, the diagnostic device(s) 710 can be and/or can include one or more implantable diagnostic, sensing, and/or monitoring device(s) or can be and/or can include one or more implantable treatment device such as an ICD, a CRT-D, a pacemaker, an implanted electrical stimulator, and/or the like. Alternatively or in addition, the diagnostic device(s) 710 can include a wearable device, such as a smart watch, a fitness tracker, an insulin pump, a thermometer, a pulse oximeter, a smart ring, and/or the like. In some embodiments, the diagnostic device(s) 710 can include a combination of devices. For example, the diagnostic device(s) 710 can include one or more implantable device(s) and one or more wearable device(s). As described in detail herein, the diagnostic device(s) 710 are configured to monitor and/or measure multiple characteristics associated with the patient P that can be used for determining and/or defining a diagnostic prediction. In some instances, the multiple characteristics can be correlated to allow for a more accurate and precise diagnostic prediction than a diagnostic prediction using just one characteristic. A brief discussion of the components of the diagnostic device(s) 710 is provided, followed by a discussion of methods and/or examples of using the system 700 and/or the diagnostic device(s) 710 thereof to generate one or more diagnoses and/or diagnostic predictions.

The diagnostic device(s) 710 include one or more sensor(s) 744 and optionally includes a processor 712, a memory 714, an input/output device 716, and a sensor interface 742. Although not shown, the diagnostic device(s) 710 can, in some embodiments, include and/or can otherwise be powered by a power system. Such a power system can include at least one battery (e.g., LiPo, Li-ion, etc.). The at least one battery can be charged when the battery is low on power. In some embodiments, the power system includes a primary cell battery and a rechargeable battery. In some embodiments, the power system can be configured to charge automatically (e.g., via patient P movement) or wirelessly (e.g., via inductive charging).

The processor 712 of the diagnostic device(s) 710 is configured to execute the operations of the diagnostic device(s) 710. In some embodiments, the processor 712 can be functionally and structurally similar to the processor 222 described above with reference to FIG. 4. For example, the processor 712 can be a hardware based integrated circuit (IC), or any other suitable processing device configured to run and/or execute a set of instructions or code such as a general-purpose processor, a CPU, an APU, an ASIC, a FPGA, a PLA, a CPLD, a PLC, and/or the like. The processor 712 can be operatively coupled to the memory 714 through a system bus (for example, address bus, data bus, and/or control bus). As described in further detail herein, the processor 712 is configured to execute instructions, code, modules, applications, etc. stored in the memory 714.

The memory 714 of the diagnostic device(s) 710 stores instructions that are executed by the processor 712. In some embodiments, the memory 714 can be functionally and structurally similar to the memory 224 described above with reference to FIG. 4. For example, the memory 714 can be any suitable volatile or non-volatile memory such as, for example, a RAM, a memory buffer, a hard drive, a ROM, an EPROM, a flash memory, and/or the like. In some instances, the memory 714 can store, for example, one or more software programs and/or code that can include instructions to cause the processor 712 to perform one or more processes, functions, and/or the like. For example, the memory 714 can include instructions that, when executed, cause the processor to process and/or analyze data received from one or more data sources (e.g., sensor data received from any number of sensors 744 and/or other data stores/sources) and determine, define, diagnose, and/or predict a state, status, event, and/or trend (e.g., a physiological and/or pathophysiological state, status, event, and/or trend) associated with the patient P.

The input/output (I/O) device 716 of the diagnostic device(s) 710 can be and/or can include any suitable device(s), interface(s), port(s), etc. that can allow the diagnostic device 710 to receive an input and/or to provide an output and/or otherwise communicate with one or more external or remote devices. For example, the I/O device 716 can be and/or can include a communications device and/or the like is configured to allow the diagnostic device(s) 710 to communicate with one or more devices such as, for example, the compute device 750, the external data source(s) 770, and/or any other suitable external device, controller, server, etc. (e.g., a compute device, controller, etc. of a medical professional, the patient P, and/or the like). The I/O device 716 can be configured to send information via one or more networks (e.g., the network(s) 705) using any suitable wired or wireless communication mode and/or device such as any of those described above.

In addition, the I/O device 716 can include a port or wireless communication device configured to communicate with and/or receive input from a keyboard, mouse, touchscreen, button, and/or any other peripheral device. In some implementations, the I/O device 716 can include a port or wireless communication device configured to communicate with and/or provide output to an audio device, a display device, a haptic device, and/or any other suitable device (e.g., external to the body of the patient P). For example, the I/O device 716 can be, can include, can be operatively coupled to, and/or can be configured to at least partially control a display that can provide at least a portion of a user interface for a software application (e.g., a mobile application, a PC application, an internet web browser, etc.) installed and/or executed on or by the compute device (or the processor thereof). In such implementations, the display can be, for example, a cathode ray tube (CRT) monitor, a liquid crystal display (LCD) monitor, a light emitting diode (LED) monitor, and/or the like.

The sensor interface 742 can be any suitable device(s), circuits, interfaces, etc. configured to allow and/or facilitate communication between the components of the diagnostic device 710 and the sensors 744. Additionally, in some embodiments, the sensor interface 742 provides the sensors 744 with power. In some embodiments, the sensor interface 742 can preprocess signals output by and/or received from the sensors 744. Alternatively, in some embodiments, the I/O device 716 can be and/or can include the sensor interface 742 and can be configured to allow communication between the sensors 744 and at least the processor 712 and/or the memory 714 of the diagnostic device(s) 710.

The sensors 744 of the diagnostic device(s) 710 are configured to sense, detect, monitor, and/or measure any number of characteristics associated with the patient P. In some embodiments, the sensors 744 include at least one sensor that is implanted into the patient P. In some embodiments, the sensors 744 include at least one sensor that is configured to sense, detect, monitor, and/or measure any number of characteristics on or outside of the body of the patient P. In some embodiments, the sensors 744 can include a sensing lead, a pressure sensor, a photoplethysmography sensor (PPG) sensor (or other optical sensor), an oxygen saturation (SpO2) sensor, an electrocardiogram (or cardio electrogram) sensor, an accelerometer, a temperature sensor, an acoustic sensor, and/or the like. Each sensor of the diagnostic device 710 can be the same type of sensor or can be a different type of sensor. For example, the sensors 744 can include multiple pressure sensors configured to measure pressure (e.g., hemodynamic pressure, etc.) at different locations on the patient P. In some embodiments, multiple sensors 744 can be co-located in or on the body of the patient P. In some embodiments, the sensors 744 are located in different locations in or on the body of the patient P. In some embodiments, at least one sensor of the sensors 744 are integrated into the diagnostic device(s) 710. In some embodiments, at least one sensor of the sensors 744 is located external to and/or apart from the diagnostic device(s) 710 (e.g., remote sensor, sensing lead, etc.) and is communicably coupled (e.g., via a wired connection, via a wireless connection, etc.) to the diagnostic device(s) 710.

In some embodiments, the sensors 744 include, at least, a first sensor configured to monitor and/or measure a first characteristic (e.g., a first bio-signal associated with and/or indicative of the first characteristic) and a second sensor configured to monitor and/or measure a second characteristic (e.g., a second bio-signal associated with and/or indicative of the second characteristic), the first characteristic being different than the second characteristic. In some embodiments, the first characteristic and the second characteristic can be related, associated, and/or the like. For example, the first and second characteristics can be related to and/or indicative of (alone or when considered together) a disease and/or other diagnoseable condition. It should be understood that the diagnostic device(s) 710 can include any number of additional sensors 744 configured to sense and/or detect any suitable characteristic(s) (and/or bio-signal(s) associated with such characteristic(s)) associated with the patient P. In some embodiments, the sensor(s) 710 can belong to a single diagnostic device(s) 710 or to multiple diagnostic device(s) 710. In some embodiments, the diagnostic device(s) 710 can generate sensor data that includes multiple signal vectors.

In some embodiments, the sensors 744 can be configured to sense, detect, monitor, and/or measure a different characteristic associated with an organ, or more generally, the patient P. In some implementations, one or more sensors 744 can be configured to sense or detect the same characteristic, thereby allowing for confirmation/verification of signal data and/or a desired degree of sensitivity and/or specificity in interpreting the signal data. In some embodiments, the sensors 744 can be configured to sense, detect, monitor, and/or measure characteristics (and/or bio-signal(s) associated with such characteristics) continuously, periodically, sporadically, or when a command is received (e.g., from the diagnostic device 710).

In some embodiments, the outputs of the sensor interface 742 and/or the sensors 744 can be calibrated based on the patient P, the position in the body of the patient P, and/or any other suitable calibration metric or combination of metrics, and/or the like. For example, the sensors 744 can be calibrated based on one or more known measurement(s) of patient characteristic(s). Calibration can include calibrating the output of the sensors 744 to align with known measurements of patient characteristics. For example, a cardiac electrical signal sensor used to measure, for example, cardiac electrical signals (e.g., electrocardiogram or cardiac electrogram) can be calibrated based on comparing and adjusting the sensor output to an external cardiac rate measuring device.

In some embodiments, the data from the sensors 744 can be used with and/or correlated with imaging data from one of more imaging devices. For example, the imaging data can include, for example, imaging data generated using coronary computed tomography angiography (CCTA), ultrasound imaging, computed tomography (CT), x-ray imaging, magnetic resonance imaging (MRI), positron emission tomography (PET) scan, and/or the like. In some embodiments, the imaging device can be used and the imaging data from the imaging device can be stored in the memory 714, a database, and/or the like. The imaging data, in turn, can be sent to the diagnostic system 700.

In some implementations, the system 700 and/or the diagnostic device(s) 710 thereof can act as and/or can form at least a portion of a remote intensive care unit (ICU). For example, the diagnostic device(s) 710 can be configured to sense, detect, monitor, and/or measure all or at least some of the characteristics that can be sensed, detected, monitored, and/or measured in an ICU including, but not limited to, cardiac cycle, pulmonary/respiratory cycle, nervous system, body temperature, glucose level, oxygen saturation, cardiac signals, sleep performance, recovery status, hemodynamic status, activity level, blood pressure, mediastinal pressure, sleep apnea, heart failure, and/or the like (or combinations and/or derivatives thereof). In some embodiments, the data provided by the diagnostic device(s) 710 (e.g., the remote ICU) can be correlated and/or used in conjunction with information and/or data stored or collected in an ICU environment in a hospital (i.e., not remote). Including the diagnostic device(s) 710 in a remote ICU environment allows for more precise diagnostics to be made in a remote setting than when only one characteristic is measured.

As described above with reference to previous embodiments, the diagnostic device(s) 710 can analyze, process, aggregate, correlate, compare, verify, confirm, etc. data received from the sensors 744 to determine and/or define a status (e.g., a physiological status, a pathophysiological status, and/or the like, or combinations thereof) associated with the patient P. Furthermore, based on the determined and/or defined status, the diagnostic device(s) 710 can detect, determine, and/or predict the occurrence of one or more health event, such as seizures; heart attacks; overdoses; tachycardia, bradycardia, and/or other arrhythmia; static and/or dynamic hyperinflation; dyspnea; and/or the like. In some embodiments, based on the determined, defined, and/or predicted status, the diagnostic device(s) 710 can determine a diagnostic status, such as a predicted diagnosis, an indication of a disease or disease state, a progression of a disease state, a condition suitable for and/or a decision to provide a given treatment/therapy (e.g., using an ICD, pacemaker, ventilator, oxygen supply, stimulator, etc.), and/or the like. In some embodiments, using the data from the sensors 744, which includes data from more than one sensor and/or otherwise associated with multiple characteristics, the status can be determined with more specification and/or accuracy than when determining a status using data from a single data source (e.g., from only one sensor detecting one bio-signal and/or characteristic).

In some implementations, the process of the diagnostic device(s) 710 (and/or the processor 712 thereof) determining the status of the patient P can include, for example, comparing, correlating, verifying, confirming, and/or synchronizing the signal data received from the sensors 744. For example, the sensors 744 can determine and/or sense a first characteristic (e.g., a first bio-signal associated with and/or indicative of the first characteristic) and a second characteristic (e.g., a second bio-signal associated with and/or indicative of the second characteristic). In some embodiments, the first characteristics and the second characteristic are different. In some embodiments, the diagnostic device(s) 710 can determine and/or sense additional characteristics and/or additional/other bio-signals. In some embodiments, the diagnostic device(s) 710 can also receive additional information associated with the patient P which can be used by the diagnostic device(s) 710 in determining the status.

The diagnostic device(s) 710 (and/or the processor 712 thereof) can determine the status based at least in part on comparing, correlating, verifying, confirming, and/or synchronizing the data from the sensors 744. To determine the status, the diagnostic device(s) 710 can, for example, determine if data associated with at least one characteristic of the characteristics measured or sensed by the sensors 744 indicates a status. Based on the correlated and/or associated characteristics, the diagnostic device(s) 710 can determine if other determined or sensed characteristics are as expected based on the status. If the characteristics are not as expected, the diagnostic device(s) 710 can modify, update, and/or augment the status. In some embodiments, one or more machine learning algorithms may be employed to define, determine, confirm, verify, correlate, predict, etc. expected status(es) and/or output(s) for a given status. In some embodiments, one or more machine learning algorithms may be employed to define, determine, confirm, verify, correlate, predict, etc. a status based at least in part on one or more relationships and/or a degree of correlation between, for example, data received from the sensors 744 (and/or any other suitable data source).

In some implementations, instructions stored in the memory 714 and executed by the processor 712 can be updated and/or refined based on learnings from previously received data. For example, data (e.g., sensor data and/or any other suitable data) can be provided as an input into one or more machine learning models to update, refine, and/or train the model(s) to provide an output associated with a predicted health condition, state, status, and/or event based on the data. In some embodiments, the compute device 750 is configured to receive the data from the diagnostic device(s) 710 and update, refine, and/or train the machine learning model(s). Once updated, the compute device 750 can send, via the network(s) 705, the updated/trained model(s) to the diagnostic device(s) 710, which in turn, can update, supplement, replace, overwrite, etc. instructions, models, algorithms, and/or processes stored in the memory 714. In some instances, the memory 714 can include the updated machine learning model, which the processor 712 can execute to process the data received from the sensors 744 or other data sources to provide a prediction and/or diagnosis. In some instances, the memory 714 can include one or more look-up tables (defined at least in part by one or more machine learning model(s)), which the processor 712 can access to select the most likely predicted state, status, event, etc. (e.g., a predicted diagnosis) based on the data.

The memory 714 can, in some embodiments, include instructions associated with preprocessing the signals from the sensor 744. For example, the memory 714 can include and/or store instructions that when executed by the processor 712 perform one or more preprocessing steps that can include amplification, filtering, analog to digital conversion, signal recognition and detection, synchronization, and/or the like. The processor 712 is configured to execute instructions, stored and/or included in the memory 714, for determining a status based on the signals, with or without preprocessing. The status can be associated with a bodily process of the patient P, a system of the body of the patient P, a portion of the body of the patient P, sensor data, and/or the like. Determining the status can include comparing an output from the sensor 744 and another output from the sensors 744 to recognize and confirm the status. For example, if the output from the sensors 744 appears to indicate an irregular heartbeat, but another output from the sensors 744 does not appear to indicate an irregular heartbeat, then the output indicating the irregular heartbeat may be determined to be a false positive. Determining and/or identifying false positives can allow the diagnostic device(s) 710 to decrease the number of undesired notifications and/or alerts for the patient P and is more specific than if using an output from just one of the sensors 744. If multiple outputs from the sensors 744 indicate the same and/or complementary status, that status is determined, corroborated, and/or confirmed.

For example, in some embodiments, the memory 714 can include instructions associated with correlating a first output from the sensors 744 and a second output from the sensors 744. In some embodiments, the first output and the second output can be complementary and confirmatory functions, characteristics, states, etc. For example, the first output can be an electrical characteristic (e.g., cardiac electrical data) and the second output can be a mechanical characteristic (e.g., cardiac mechanical data and/or any other mechanical data sensed or detected within a portion of the body of the patient P such as the anterior mediastinum), which can allow the processor 712 to correlate and/or verify an electrical characteristic with or to an expected mechanical characteristic. As another example, the first output can be respiratory data (e.g., respiratory rate, period, cycle, etc. as determined and/or sensed by a pressure sensor and/or the like), and the second output can be oxygen saturation data (e.g., from an internal or external pulse oximeter and/or the like). In some embodiments, the memory 714 can include instructions associated with correlating the outputs from the sensors 744 with imaging data generated and/or received from the external data source(s) 770.

As described above, the process of determining a status of the patient P can utilize and/or otherwise employ one or more artificial intelligence algorithm(s) (e.g., a machine learning model(s) and/or algorithm(s)) to determine and/or predict the status. For example, the machine learning model(s) can be trained to determine the status based at least in part on outputs from the sensors 744. In some embodiments, the compute device 750 can be used to define the machine learning models and/or to perform the training, refining, etc. In some embodiments, the machine learning model(s) and/or algorithm(s) (referred to herein as machine learning “model” or “algorithm” interchangeably) can be configured to filter out or recognize physiological activity (e.g., breathes, heartbeats, physical activity, etc.) that were determined or missed during an initial determination. In some embodiments, the machine learning algorithm(s) can correlate, compare, verify, confirm, and/or otherwise process the outputs from sensors 744 to determine a pattern corresponding to a status, etc. In some embodiments, the machine learning algorithm(s) can learn from the outputs of the sensors 744 to determine long-term correlation(s), patterns, trends, and/or changes thereof between the outputs of the sensor 744.

In some embodiments, the process of determining the status can utilize and/or otherwise employ one machine learning model (e.g., a multivariate model). In some embodiments, the process of determining the status can utilize and/or otherwise employ more than one machine learning model. In some embodiments, different outputs from the sensors 744 can be processed by different machine learning models. For example, a first output from a first sensor (e.g., including data associated with cardiac electrical signals radiating from the heart) can be processed by a first machine learning model and a second output from a second sensor (e.g., including data associated with, for example, pressure changes in an anterior mediastinum of the patient P) can be processed by a second machine learning model. In some embodiment, a machine learning model can be associated with multiple types of sensor outputs. In some embodiments, information from the external data source(s) 770 can also be processed (independently or in conjunction with any other model) by a machine learning model. In some embodiments, each machine learning model can be configured to output, based on the data it receives/processes, a prediction associated with a status of the patient. In some embodiments, the compute device 750 can be used to define, train, and/or refine the machine learning models and/or to define, train, and/or refine one or more associations, correlations, aggregations, etc. of the outputs of multiple machine learning models. As such, steps that involve to use of a high degree of processing power can be performed and/or executed by the compute device 750, while the diagnosis device(s) 710 can perform steps that implement and/or use the previously defined models, which generally do not require significant processing power (and therefore, are suitable for execution by a device implanted in the body of the patient P.

In some embodiments, a machine learning model can be used to correlate, confirm, and/or verify the outputs from the sensors 744. In some embodiments, the machine learning model used to correlate the outputs from the sensors 744 may be different than the machine learning model(s) used to initially analyze and/or process the output from the sensors 744. In some embodiments, a machine learning model can be used to aggregate the correlated data. In some embodiments, the machine learning model used to aggregate the outputs from the sensors 744 may be different than the machine learning model(s) used to initially analyze and/or process the output from the sensors 744 and to correlate the data. For example, determining the status can include a series of machine learning models that are used to process data from the sensors 744, correlate the processed data, aggregate the sensor data, and then determining the status based on the data.

In some embodiments, the machine learning models used in determining the status can be the same type of machine learning model. In some embodiments, at least two of the machine learning models used in determining the status can be different machine learning models. For example, the machine learning models can include a support vector machine, deep learning, convolution neural network, recurrent neural network, regression, and/or the like. The type of machine learning model used can be associated with the type of data being processed, the source, and/or the like. For example, it may be desirable to process raw data from the sensors 744 with a first type of machine learning model and to correlate the outputs from the sensors 744 with a second type of machine learning model. As another example, it may be desirable to process the data from the sensors 744 with a first type or first group of machine learning model(s) and to correlate the outputs from the first type or group of machine learning model(s) using a second type or second group of machine learning model(s).

In some embodiments, the machine learning algorithm(s) described herein can use weights, scores, or similar metrics to determine the diagnostic status and/or to make a diagnostic prediction. For example, certain types of data, data from certain sources, data from different sensor 744 locations, data from different sensor 744 types, and/or the like can have a higher or otherwise different score than other data. In some embodiments, the weights, scores, or similar metrics may be associated with the relevance of the data or the predictive value of the data (or at least expected predictive value). For example, data associated with characteristics related (e.g., directly) to the status being determined can have a higher score, where a higher score indicates a higher relevance, than characteristics that are less related (e.g., indirectly). For example, if the status is a cardiac status, direct or inferred cardiac electrical and/or mechanical measurements can have a higher score than, for example, a temperature measurement. Alternatively, if the status is not directly related to the heart, cardiac electrical and/or mechanical measurements may be less determinate and therefore, given a lower score or weight.

In some embodiments, the machine learning models can be configured to cluster relevant signals from the sensors 744 (and/or any other data source). For example, the machine learning models can be configured to cluster signals that are relevant to a status or multiple statuses (e.g., physiological status(es), pathophysiological status(es), and/or the like, or combinations thereof) that the diagnostic device(s) 710 are configured to determine. Clustering signals can allow for the diagnostic device(s) 710 to only process and/or correlate data and/or signals from the sensors 744 and/or the external data source(s) 770 that is relevant to the desired status(es). For example, if a user indicates that only certain status determinations are desired, the diagnostic device(s) 710 may only process the data that has been clustered as being relevant to the status indicated as desired.

In some embodiments, the machine learning models can be configured to use image data from an imaging device (e.g., coronary computed tomography angiography (CCTA) imaging data, and/or any other suitable image data) to improve and/or correlate predictions and/or diagnoses based on data from the sensors 744. Alternatively, data output by the sensors 744 (and/or predictions and/or diagnoses based on the data) can be used to improve and/or correlate determinations, inferences, predictions, diagnoses, etc. based on the image data. The image data can be used to determine if a condition is occurring that other sensors may not indicate. For example, the image data can show if an artery is blocked, if a tumor is present (or show characteristics associated with a tumor), if a bone is damaged or broken, and/or the like. In some embodiments, machine learning algorithm(s) can be used to correlate and/or augment the data from the sensors 744 with the image data. In some embodiments, the machine learning algorithm(s) can be used to make predictions on what the image data is showing and how it relates to a status.

In some embodiments, the memory 714 can store instructions that cause the processor 712 to determine whether a health event (e.g., an adverse health event that may be undesirable or dangerous to the patient P) is occurring based on the status or predicted status of the patient P. In some embodiments, the processor 712 may execute a machine learning algorithm, which may be the same or different than that used in refining, determining, and/or predicting the status, for determining if a health event is occurring. The memory 714 can further include and/or store instructions for execution by the processor 712 associated with determining the type of health event. For example, the processor 712 determining the type of health event can include correlating the status with known statuses associated with health events. In some embodiments, the memory 714 can further include and/or store instructions for determining if the health event is an adverse health event. Similarly stated, the memory 714 can include and/or store instructions for determining and/or distinguishing an adverse health event from, for example, a normal or non-adverse health event that may present and/or may be associated with one or more similar characteristics. In some embodiments, the processor 712 can determine the type of adverse health event (e.g., any of the adverse health events described herein). In some embodiments, the processor 712 may execute one or more machine learning algorithms, which may be the same or different than the other machine learning algorithms described herein, for determining the type of health event.

In some embodiments, the memory 714 can store instructions that cause the processor 712 to determine and/or define a diagnosis and/or diagnostic prediction (e.g., a result of a diagnostic test of the patient P) based on the status, health event, and/or additional information associated with the patient P. In some embodiments, the processor 712 may execute a machine learning model, which may be the same or different than that used in refining, determining, and/or predicting the status, for determining if a health event is occurring. In some embodiments, the diagnostic device(s) 710 can be used for both determining if a health event is occurring and for determining a diagnostic status or one of determining if a health event is occurring or for determining a diagnostic status.

While the memory 714 is described above as storing one or more machine learning models (executed by the processor 712), which can be used to determine the occurrence of a health event and/or to make a diagnosis or a diagnostic prediction for a patient, in other implementations, the memory 714 can store any number of processes, functions, instructions, etc. (executed by the processor 712) that is/are not machine learning model(s). In some implementations, for example, any of the machine learning models described herein can be used to determine and/or define an algorithm and/or a set of relationships, correlations, etc. for processing data associated the characteristic(s) (e.g., physiological characteristic(s), pathophysiological characteristic(s), and/or the like, or combinations thereof). In this manner, the processor 712 can execute the defined and/or determined algorithm without having to execute the machine learning model(s), which in turn, may reduce processing load and/or time. In other implementations, a look-up table may be defined (e.g., using any of the machine learning models described herein and/or via any other suitable process). In such implementations, the processor 712 can reference the look-up table to determine, based on the data associated with the characteristic(s), a diagnostic status for the patient and/or whether a heath event is occurring (or a likelihood thereof).

In some embodiments, the memory 714 can store instructions that cause the processor 712 to generate a notification associated with the status, health event, and/or the diagnostic status. The notification can be used to indicate to a user of a status for monitoring purposes, or how a status relates to a predetermined threshold. The notification can be used to indicate to a user that a health event is occurring. The notification can be used to indicate to a user that the diagnostic status is ready for review. In some embodiments, the notification can be configured for sending to an emergency responder, a medical provider, secondary user (e.g., other than the patient P), and/or the like. The notification can be used so that corrective action can be taken if necessary.

In some embodiments, the memory 714 can store instructions that cause the processor 712 (and/or the I/O device 716) to indicate, and/or generate data indicating, that a health event is occurring. For example, the diagnostic device 710 (or processor 712 and/or the I/O device 716 thereof) can define data or one or more signals associated with a visual, auditory, and/or physical notification that indicates that the health event is occurring. For example, diagnostic device 710 can cause and/or instruct a display to display that the health event is occurring, an alarm to sound or other be graphically represented on the display, a vibration to occur, and/or the like. Similarly, the diagnostic device 710 can cause and/or can instruct the display to represent results from and/or data associated with a diagnostic test. For example, the display can display a value associated with a diagnosis and/or an indication that a diagnostic test is complete, a light can indicate that a diagnostic status, a vibration can indicate a diagnostic status, and/or the like. In some implementations, one or more signals, notifications, and/or the like can be sent (e.g., via the network(s) 705) to the compute device 750. For example, the diagnostic device 710 can be configured to send a signal indicative of an alarm notification to the compute device 750. The alarm notification can indicate a type of health event, such as a heart attack, seizure, overdose, etc., so that a user associated with the compute device 750 can respond to the health event.

In some embodiments, the diagnostic device 710 can receive input signals and/or commands from a device external to the body of the patient P and can, in turn, provide the input signals to the processor 712 of the diagnostic device(s) 710. For example, the diagnostic device 710 (and/or the I/O device 716 thereof) can receive input signals from one or more devices (e.g., touchscreen, keyboard, mouse, buttons, etc.) representing inputs by a user (e.g., doctor, patient, caretaker, etc.). The inputs, in turn, can represent information associated with a diagnostic status, the patient P, a health event, and/or the like. In some embodiments, if the user receives a notification that a health event is occurring (e.g., from or generated by the I/O device 716), the user can indicate that an alarm system should be sent to an emergency responder, medical professional, and/or the like. In some embodiments, the diagnostic device 710 can be configured to receive data associated with the patient P such as from the external data source(s) 770 and/or the compute device 750, which may alter and/or may otherwise be used in the operation of the diagnostic device(s) 710 and/or in the determination of a diagnosis or in a treatment decision, etc.

In some implementations, the diagnostic device 710 can send information to a user and/or a compute device controlled by a user regarding the diagnostic device(s) 710 and/or an operational status of the diagnostic device(s) 710 such as battery level, notifications, device status, and/or the like. The diagnostic device 710 can also send information regarding the patient P, such as real-time or substantially real-time information, data, and/or other signals associated with the patient P (e.g., data from the sensors 744, and/or any other suitable data). In some embodiments, the diagnostic device 710 can receive signals for augmenting and/or at least partially controlling the operation of the diagnostic device(s) 710. For example, the diagnostic device 710 can receive signals (e.g., from the compute device 750, the external data source(s) 770, and/or any other device) associated with patient information and/or other operational instructions for determining health events, diagnoses, and/or the like. In some embodiments, the diagnostic device 710 can be configured to send information and/or signals to the compute device 750 and/or server that is/are configured to perform machine learning processes. The diagnostic device 710 can also receive information from the compute device 750 and/or the server that may include instructions, firmware, updates, etc. based on an output from a machine learning model.

In some embodiments, the compute device 750 described above may be configured to execute and/or perform any of the processes and/or any suitable portion of the processes associated with the system 700. For example, certain operations described as being performed by the diagnostic device(s) 710 can be executed by the compute device 750. In some implementations, the diagnostic device(s) 710 may be configured to preprocess data from the sensors 744 while the compute device 750 is configured to process, correlate, and/or the like the preprocessed data.

In such implementations, the compute device 750 can be used to complete certain processes that may be too resource intensive (e.g., battery power, memory, processing power, etc.) for the diagnostic device(s) 710 and/or to otherwise decrease the computational resources needed by the diagnostic device(s) 710. In some embodiments, for example, the compute device 750 can be used to define, train, execute, and/or update, via the processor 752, one or more of the machine learning models, processes, and/or algorithms described above. In such embodiments, the compute device 750 can receive data from a large number of patients and/or large amounts of data from, for example, the external data source 770, which in turn can be used to train the machine learning models, processes, and/or algorithms. Once defined, trained, updated, etc., the machine learning models, processes, and/or algorithms, can be executed by the diagnostic device(s) 710 using a given set of data associated with a given patient P. In this example, the amount computational resources associated executing the machine learning model for a given set of data associated with the patient P is significantly less than the computational resources associated with defining, training, updating, and/or refining the machine learning model.

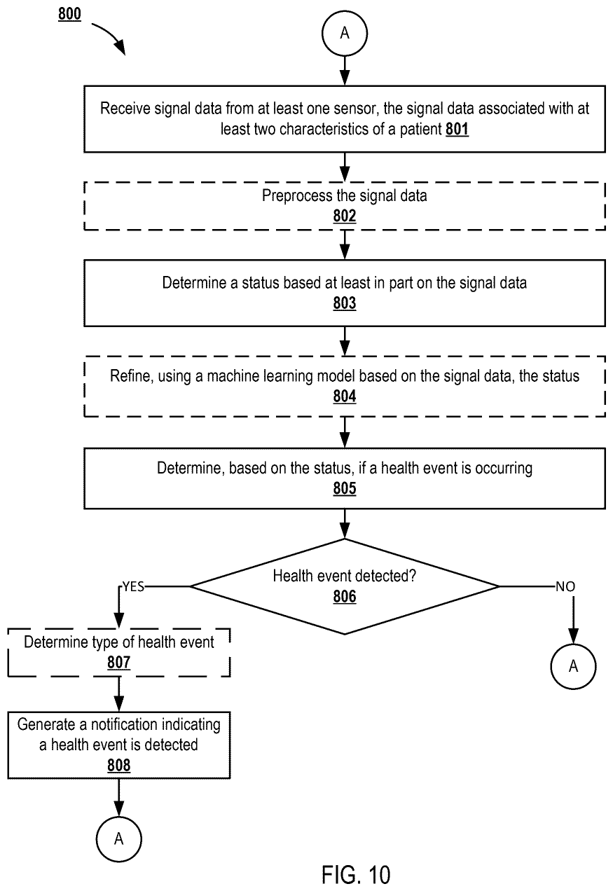

FIG. 10 is a flow chart depicting a method 800 for using a diagnostic system, according to an embodiment. In some embodiments, the method 800 can be executed and/or performed by a diagnostic/treatment system that is functionally and/or structurally similar to the diagnostic/treatment system 700 of FIG. 9. In some embodiments, the method 800 can be executed by one or more devices. In some embodiments, the method 800 is configured to use at least two signals form at least two data source(s) (e.g., sensors) to determine if a patient is having a health event (e.g., sudden health event, chronic health event, etc.). Using at least two signals reduces the likelihood of that a determining that a health event is occurring is a false positive. The method 800 includes generating a notification indicating that the health event is detected, which allows for a patient or a user to review the health event and take corrective action. The method 800 can be used by a patient that may be at-risk of a health event and is monitoring for a health event, thus allowing the patient and/or a caretaker or healthcare provider to act on the health event if the health event is determined to be occurring.

The method 800 includes, at 801, receiving signal data from at least one sensor (e.g., functionally and/or structurally similar to any of the sensors described herein). The signal data is associated with at least two characteristics of a patient. In some embodiments, the at least one sensor can include any number of sensors. In some embodiments, the at least two characteristics can include a first characteristic and a second characteristic. The first characteristic can be associated with a status (e.g., physiological status(es), pathophysiological status(es), and/or the like, or combinations thereof) and the second characteristic can be associated with the same or different status. For example, the at least two characteristics can include an electrical characteristic associated with a status and a mechanical characteristic associated with the status. As another examples, the at least two characteristics can include a characteristic (electrical, mechanical, etc.) associated with a first status and a characteristic (electrical, mechanical, etc.) associated with a second status different from the first status. In some embodiments, step 801 may include receiving additional signal data from any number of additional sensors and/or any additional or other data sources (e.g., one or more ex vivo data sources). In some embodiments, step 801 may include receiving the signal data from an external data source (e.g., patient database, etc.). In some embodiments, step 801 may additionally include receiving additional information associated with the patient such as patient age, height, weight, demographic information, medical history, and/or the like.

The method 800 optionally includes, at 802, preprocessing the signal data. Preprocessing can include noise reduction, filtering, amplification, normalization, conversion (e.g., analog-to-digital, etc.), synchronization, and/or the like. Preprocessing can prepare for the signals to be received by another component of the diagnostic/treatment system. For example, a sensor interface (e.g., functionally and/or structurally similar to any of the sensor interfaces described herein) can preprocess the signal data so that the signal data can be received by the compute device (e.g., functionally and/or structurally similar to any of the compute devices described herein). In some embodiments, the compute device may preprocess the first signal data and/or the second signal data to prepare the data for processing.

The method 800 includes, at 803, determining a status based at least in part on the signal data. The status can correspond to the status of a portion of the patient such as the cardiac system and/or pulmonary/respiratory system. In some embodiments, determining a status can further be based on data from additional sources (e.g., additional sensors and/or one more ex vivo data sources). Determining cardiac status can include, for example, comparing and synchronizing the signal data and/or artifacts in the signal data to determine the status or functioning of the portions of the patient P. For example, when signal data associated with a first characteristics (e.g., first signal data, electrical signal data) is associated with electrical signals and the signal data associated with the second characteristic (e.g., second signal data, mechanical signal data) is associated with mechanical signals, determining status can include synchronizing an electrical signal curve (from the first signal data) and a mechanical signal curve (from the second signal data) to correlate the signal data. As another example, determining the status can include determining if features seen in the electrical signal data have an expected corresponding result in the mechanical signal data. As such, using data associated with more than one characteristic provides more sensitivity and specificity in determining cardiac status than when determining cardiac status using just one source of data (e.g., electrical signals alone).

At 804, the method 800 optionally includes refining the cardiac status using a machine learning model based on the signal data. Refining can include, for example, improving, tuning, updating, and/or verifying the status determined at 803. The machine learning model can be trained based on status associated with data from the at least one sensor. For example, the machine learning model can be trained based on status associated with electrical signal data and mechanical signal data from the patient, historical data, data from studies, medical professional input, and/or the like. In some embodiments, the machine learning model can be trained based on additional patient data (e.g., activity level, height, weight, age, and/or the like). Training on patient data allows for the machine learning model to gain further insight on the patient that may affect the characteristics of the patient measured by the sensors.

In some embodiments, the status may be changed, updated, tuned, etc., based at least in part on comparing the cardiac status from step 803 to more closely align with empirical data, a status determination by one or more trained cardiologists, updated clinical evidence, and/or any suitable known or accepted ground truth. In some embodiments, the machine learning model may be configured to recognize long-term changes in status and/or the signal data and adapt to these changes. For example, the machine learning model may recognize that the mechanical signal data has been decreasing over weeks, months, or years. Recognizing changes allows the machine learning model to adapt the sensitivity along with the patient as the patient changes. In some embodiments, the machine learning model may receive feedback data and/or training sets that are used to further train the machine learning model.

The method 800 includes, at 805, determining, based on the status, if a health event is occurring. Determining if a health event is occurring can include, in some embodiments, comparing the status to an expected status. For example, if the status indicates that a characteristic is higher than it expected, the presence of a health event may be determined. In some embodiments, a machine learning model can be used to determine if a health event is present based at least in part on the status. For example, the machine learning model can recognize changes in the patient's activity and/or characteristics that can indicate the presence of a health event, or if no health event is occurring. In some embodiments, an indication that a health event is occurring may be sent to the user. In some embodiments, the machine learning model can recognize when the patient's activity is expected and/or predicted to increase. For example, when the patient is exercising, experiencing stress or anxiety, and/or the like. For example, if the sensors include an accelerometer, a determination that the patient's activity is greater than expected can be made when an acceleration value, acceleration vector, change in an acceleration vector, and/or the like indicates that the patient is active, was active, or will be active.

The method 800 includes, at 806, determining if a health event is detected. At 806, the method 800 includes a decision of whether a health event has been detected. If a health event has not been detected, the method 800 returns to step 801 to continue monitoring the heart of the patient. If a health event has been detected, the method 800 continues to step 807.

The method 800 optionally includes, at 807, determining the type of health event. In some embodiments, determining a type of health event can include comparing and matching the status to known health events. In some embodiments, a machine learning model may be used to match the status to a health event. In some embodiments, the type of health event can be sent to a user device and/or the like for review by the user. For example, the type of health event can be reviewed by the patient, a medical proxy, a medical professional, and/or the like. In some embodiments, the type of health event may include a severity score. The severity score can indicate an urgency and/or a health danger associated with the health event. The method 800 includes, at 808, generating a notification indicating a health event is detected. In some embodiments, the notification is generated in response to the health event having a severity score that indicates an urgent health event. In addition to an indication that a health event is occurring, the notification can include additional information about the health event, such as the type of health event, the characteristics associated with the health event, and/or the like. The notification can be sent to an external device, such as a user device, a healthcare provider, an emergency responder, and/or the like. In some embodiments, the notification can be displayed on a wearable. In some embodiments, an indication of the notification can be displayed on the wearable and further information can be displayed on the user device.

While the method 800 is described above as generating a notification associated with a positive detection of a health event, the method 800 may include generating notifications associated with any suitable data and/or determination in addition to and/or as an alternative to the generation of the notification indicating the health event is detected (at step 808). For example, such notifications may be indicative of any determination, inference, calculation, and/or monitoring of data received from any number of sensors. Similarly stated, the generation of one or more notifications need not be limited to the determination of a given health event. Rather, the method 800 may include generating a notification indicating any suitable health event, status, characteristic, determination, inference, trend, correlation, etc. (or one or more changes associated therewith).

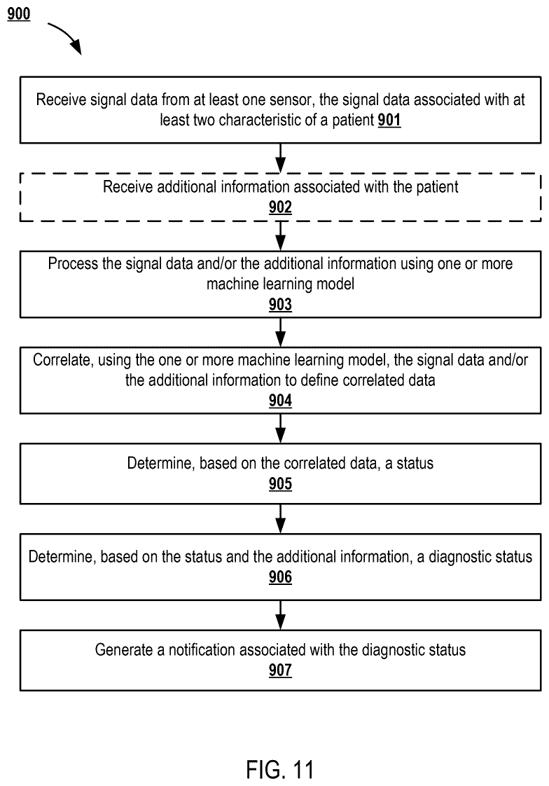

FIG. 11 is a flow chart depicting a method 900 for using a diagnostic/treatment system to, for example, determine a diagnostic status of a patient, according to an embodiment. In some embodiments, the method 900 can be executed and/or performed by a diagnostic/treatment system that is functionally and/or structurally similar to the diagnostic/treatment system 700 of FIG. 9. In some embodiments, the method 900 can be executed by one or more devices. In some embodiments, the method 900 is configured to use at least two signals from at least two data source(s) (e.g., sensors) to determine a diagnostic status of a patient. Using at least two signals reduces the likelihood that a determined diagnostic status is a false positive. The method 900 includes generating a notification indicating the diagnostic status, which allows for a patient or a user to review the health event and take corrective action. The method 900 can be used in a diagnostic environment which can include an environment where health issues and diagnoses are desired such as in a clinical setting, a hospital, a remote ICU, and/or the like.

The method 900 includes, at 901, receiving signal data from more than one sensor (e.g., functionally and/or structurally similar to the sensors 744). The signal data is associated with at least one characteristic of the patient. In some embodiments, the at least one sensor can include any number of sensors. In some embodiments, the at least two characteristics can include a first characteristic and a second characteristic. The first characteristic can be associated with a status and the second characteristic can be associated with the same or different status. In some instances, the characteristics and/or the status(es) can be associated with physiological characteristic(s) and/or status(es), pathophysiological characteristic(s) and/or status(es), and/or combinations thereof. For example, the at least two characteristics can include an electrical characteristic associated with a status and a mechanical characteristic associated with the status. As another examples, the at least two characteristics can include a characteristic (electrical, mechanical, etc.) associated with a first status and a characteristic (electrical, mechanical, etc.) associated with a second status different from the first status. In some embodiments, step 901 may include receiving additional signal data from any number of additional sensors and/or additional data sources (e.g., one or more ex vivo data sources). In some embodiments, step 901 may include receiving the signal data from an external data source (e.g., patient database, etc.). At 902, the method 900 may optionally include receiving additional information associated with the patient. The additional information can include information associated with the patient such as patient age, height, weight, demographic information, medical history, and/or the like.

The method 900 includes, at 903, processing the signal data and/or the additional information using one or more machine learning model. Processing can include using the one or more machine learning model to refine the signal data and/or the additional information. In some embodiments, the machine learning model can be configured to filter out or recognize activity (e.g., breathes, heartbeats, physical activity, etc.) that were determined or missed during an initial determination. In some embodiments, signal data associated with different characteristics and/or sensor can be processed by different machine learning models. For example, a signal data associated with a first characteristic can be processed by a first machine learning model and signal data associated with a second characteristic can be processed by a second machine learning model. In some embodiment, a machine learning model can be associated with multiple types and/or sources of signal data. In some embodiments, the additional information can be associated with a machine learning model that is configured to process the information.

In some embodiments, the one or more machine learning models can be configured to cluster signal data and the additional information based on relevance to a known status. For example, the machine learning models can be configured to cluster signals that are relevant to a status or multiple statuses that the diagnostic/treatment system is configured to determine. Clustering signals can allow for later correlation (e.g., at 904) of the signals that are relevant to the desired status(es). For example, if a user indicates that only certain status determinations are desired, clustering may cluster only the signal data and the additional information that is relevant to the status indicated as desired.

The method 900 optionally includes, at 904, correlating, using the one or more machine learning model, the signal data and/or the additional information to define correlated data. In some embodiments, the machine learning model can correlate the signal data and/or the additional information to determine a pattern. In some embodiments, the one or more machine learning models can learn from the outputs of the at least one sensor to determine long-term correlation and changes in correlation between the outputs of the at least one sensor. In some embodiments, the machine learning model used to correlate the signal data and/or the additional informant may be different than the machine learning model(s) used to initially process the output from the signal and/or the additional information. In some embodiments, the machine learning model used to aggregate the outputs from the sensors may be different than the machine learning model(s) used to process the signal data and/or the additional information at 903.

The method 900 includes, at 905, determining, based on the correlated data, a status. In some embodiments, determining the status can utilize one machine learning model. In some embodiments, determining the status can utilize more than one machine learning model. The status can correspond to the status of a portion of the patient such as the cardiac system and/or pulmonary/respiratory system. In some embodiments, determining a status can further be based on data from additional sources (e.g., additional sensors and/or one more ex vivo data sources). Determining cardiac status can include, for example, comparing and synchronizing the correlated data and artifacts in the signal data to determine the status or functioning of the portions of the patient. As another example, determining the status can include determining if features seen in the electrical signal data have an expected corresponding result in the mechanical signal data. As such, using data associated with more than one characteristic provides more sensitivity and specificity in determining cardiac status than when determining cardiac status using just one source of data (e.g., electrical signals alone).

The method 900 includes, at 906, determining, based on the status and the additional information, a diagnostic status. Determining the diagnostic status can include matching the status with a known diagnosis, or lack thereof. For example, a machine learning model can be used to determine if the status is associated with a known diagnosis. The method 900 includes, at 907, generating a notification associated with the diagnostic status. The notification can be used to indicate to a user of the diagnostic status for monitoring purposes and/or treatment purposes. The notification can be used to indicate to a user that the diagnostic status is ready for review. In some embodiments, the notification can be configured for sending to an emergency responder, a medical provider, secondary user (e.g., other than the patient P), and/or the like. The notification can be used so that corrective action can be taken if necessary.

While the method 900 is described above as generating a notification associated with a positive detection or determination of a diagnostic status, the method 900 may include generating notifications associated with any suitable data and/or determination in addition to and/or as an alternative to the generation of the notification associated with the diagnostic status (at step 907). For example, such notifications may be indicative of any determination, inference, calculation, and/or monitoring of data received from any number of sensors. Similarly stated, the generation of one or more notifications need not be limited to the determination of a given diagnostic status. Rather, the method 900 may include generating a notification indicating any suitable health event, status, characteristic, determination, inference, trend, correlation, etc. (or one or more changes associated therewith).

While the methods 800 and 900 are described above as determining the occurrence of a health event and/or a diagnostic status for a patient, in some implementations, the determination of the health event and/or diagnostic status need not be determined after receiving data from the one or more sensors and/or data sources. For example, in some implementations, data received from the one or more sensors and/or data sources may be stored for a period of time prior to being used in the determination of the health event and/or diagnostic status. In some implementations, the data may be stored locally (e.g., in a memory of the diagnostic device) and then sent to an external device for processing and/or analysis in addition to and/or as an alternative to the determination of the health event and/or diagnostic status. In some implementations, the determination of the health event and/or diagnostic status is based on data received from one or more sensors and/or data sources over a period of time (e.g., an extended period of monitoring and/or the like) during which the diagnostic device can store the data received prior to analyzing. In some implementations, the determination of the health event and/or diagnostic status can be retrospective based on an analysis and/or processing of data received over a period of time and stored, for example, in a memory of the diagnostic device. In some implementations, such a retrospective analysis can be performed on data associated with and/or otherwise from any number of diagnostic devices (e.g., from the diagnostic devices of multiple patients).

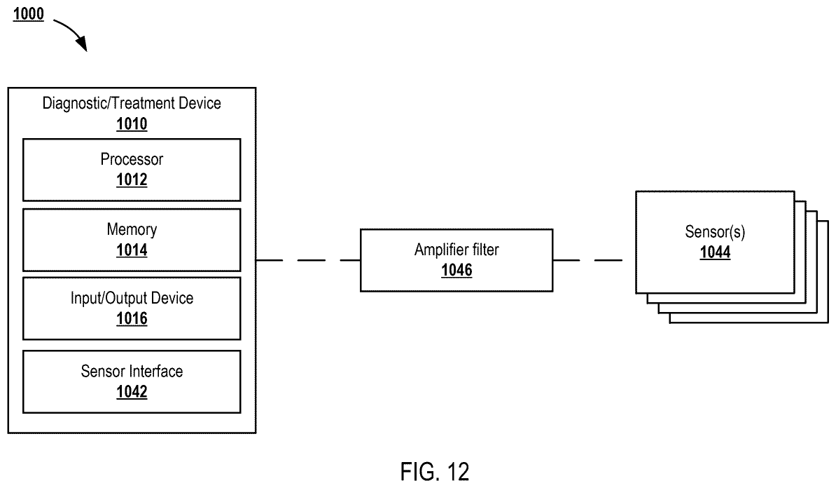

FIG. 12 is a schematic illustration of a diagnostic/treatment system 1000 including a diagnostic/treatment device 1010 operably coupled to and/or configured to be operably coupled to one or more amplifier/filters 1046 and sensors 1044, according to an embodiment. Similar to the diagnostic/treatment devices described above (e.g., the diagnostic/treatment device 110 of FIG. 3), the diagnostic/treatment device 1010 can be an implantable medical device configured to sense, detect, monitor, and/or measure one or more bio-signals and/or characteristics of the patient and to use data associated with the bio-signals and/or characteristics to provide diagnostic functionality and/or predictions and/or provide therapeutic/treatment decision-making, which in turn, can be carried out and/or delivered by one or more portions of the diagnostic/treatment device 1010.

As shown, the diagnostic/treatment device 1010 includes a processor 1012, memory 1014, an input/output (I/O) device 1016, and a sensor interface 1042. The processor 1012, the memory 1014, the I/O device 1016, and the sensor interface 1042 can be similar to and/or substantially the same as any of the processors, memories, I/O devices, and sensor interfaces, respectively, included in any other diagnostic/treatment devices described herein. Accordingly, the processor 1012, the memory 1014, the I/O device 1016, and the sensor interface 1042 are not described in further detail herein.

At least one amplifier/filter 1046 is included in and/or coupled to the diagnostic/treatment device 1010. A sensor or multiple sensors 1044 may couple to the at least one amplifier/filter 1046. The amplifier/filter 1046 shown may be a single amplifier/filter, a cascade or multitude of amplifier/filters, a combination of an amplifier and a filter, multiple combinations of amplifiers and filters, a digital amplification and filtering process performed by the processor 1012, and/or the like. In some embodiments, the amplifier/filter 1046 can be and/or can include one or more high-pass or low pass filters that can be configured to filter the pressure data based at least in part on frequency. In some embodiments, the amplifier/filter 1046 can be and/or can include any suitable device configured to amplify a magnitude of at least a portion of the data representing pressure changes in the mediastinal space. Such amplification can allow for a greater degree of sensitivity in detecting pressure changes.

In operation, at least one sensor 1044 of the diagnostic/treatment device 1010 senses, detects, monitors, and/or measures one or more bio-signals which is/are then amplified and filtered, via the amplifier/filter 1046, for use by the diagnostic/treatment device 1010. The signals sensed, detected, monitored, and/or measured by the sensor(s) 1044 are fed into the amplifier/filter 1046, which filters, separates, and amplifies one or more portions of the signals. The amplified and filtered/separated signals are used for the purpose of diagnosing, or for feedback when treating, as discussed with FIG. 3.

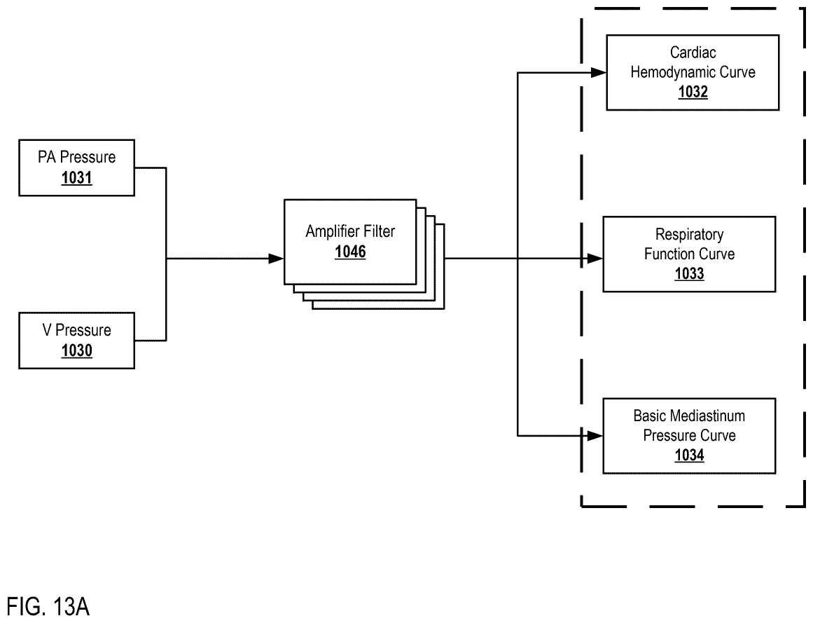

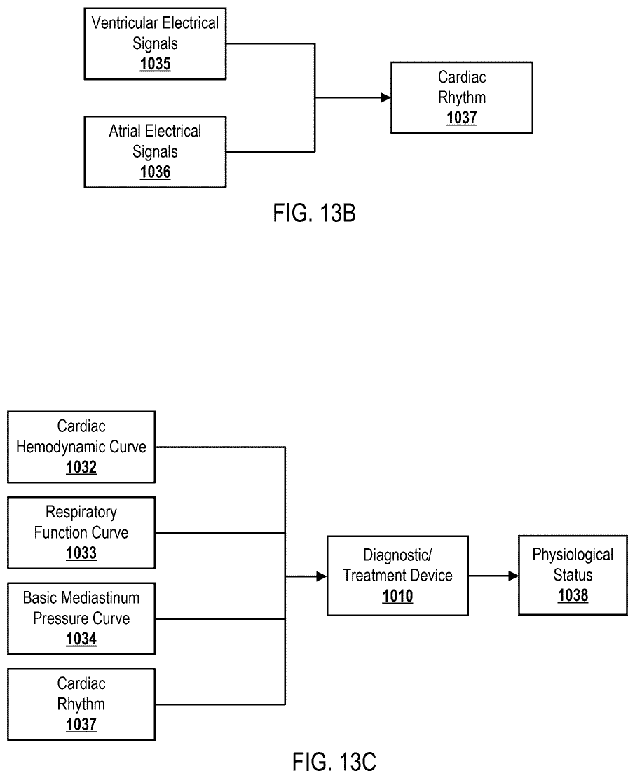

FIGS. 13A-13C are schematic illustrations of an algorithm (or portions thereof), performed by the diagnostic/treatment device 1010 of FIG. 12, associated with processing sensed, detected, monitored, and/or measured pressures into different pressure curves. For example, the sensors 1044 of FIG. 12 can be implanted in the body of a patient and can collect data and/or otherwise measure pressure within the anterior mediastinum. As shown in FIG. 13A, the pressure data can include, for example, Ventricular pressure 1030 (“V pressure 1030”) and pulmonary artery pressure 1031 (“PA pressure 1031”). The pressure signals are sensed, detected, monitored, and/or measured and are sent through the amplifier/filter 1046 to separate the signals into distinguishable and/or distinct curves. In the example of FIG. 13A, the curves generated from the PA pressure 1031 and V pressure 1030 measurements are a cardiac hemodynamic curve 1032, a respiratory function curve 1033, and a basic mediastinum pressure curve 1034. The pressure curves can be further digitally filtered to obtain more stable and precise pressure curves.

In some embodiments, the amplification and/or filtering, performed by the amplifier/filter 1046 can be based at least in part on differing frequencies within the pressure data. For example, pressure changes due to respiration can have a relatively low frequency, while pressure changes due to cardiac activity (e.g., the beating of the heart) can have a relatively have frequency. These frequencies, for example, correspond to and/or are indicative of the underlying physiological processes producing the pressure changes. For example, a normal respiration rate for an adult patient at rest can be around 12-20 breaths per minute, while a normal heart rate for an adult patient at rest can be around 60-100 beats per minute. Thus, pressure changes associated with cardiac function can have a frequency about 5 times faster than a frequency associated with respiratory function. In some instances, pressure changes associated with the basic mediastinal pressure (or background pressure) can take place over an extended period and thus, a frequency of changes to the basic/background mediastinal pressure can have a very low frequency compared to the frequencies associated with respiration and/or cardiac function.

As described above with reference to previous embodiments, one or more sensor 1044 of the diagnostic/treatment device 1010 can be configured to sense, detect, monitor, and/or measure cardiac electrical signals radiating from the heart. In some embodiments, the sensor(s) 1044 configured to detect and/or monitor cardiac electrical signals can be and/or can include one or more electrodes and/or the like, which can be included in and/or positioned along one or more leads (not shown) of the diagnostic/treatment device 1010. As shown in FIG. 13B, cardiac electrical data can include, for example, ventricular electrical signals 1035 and atrial electrical signals 1036. These electrical signals are fed back to the processor 1012, which can process and/or analyze the data and output, for example, data associated with the cardiac rhythm 1037 of the patient. Although not shown in FIG. 13B, in some embodiments, the diagnostic/treatment device 1010 can include one or more amplifier/filters configured to amplify and/or filter the electrical signals.

The relationship between the waveform morphology of the cardiac pressure curve and simultaneously recorded waveforms of the cardiac electrical signals can reflect many changes in cardiac function and hemodynamic parameters. The respiratory pressure curve can reflect respiratory rate and rhythm changes, respiratory movement, and respiratory function. The basic mediastinal pressure curve can reflect long-term, slow mediastinal pressure changes, fluid status, chronic cardiac function changes, heart failure decompensation for both low ejection fraction and preserved ejection fraction patients, and mediastinal diseases.

In some implementations, data associated with and/or indicative of the cardiac hemodynamic curve 1032, the respiratory function curve 1033, and the basic mediastinum pressure curve 1034 (FIG. 13A), and data associated with and/or indicative of the cardiac rhythm 1037 (FIG. 13B) are all fed back to the processor 1012 of the diagnostic/treatment device 1010 and at least temporarily stored in the memory 1014. As shown in FIG. 13C, the diagnostic/treatment device 1010 can process the data associated with and/or indicative of the cardiac hemodynamic curve 1032, the respiratory function curve 1033, the basic mediastinum pressure curve 1034, and the cardiac rhythm 1037 to determine and/or define physiological status 1038 of the patient. through usage of a diagnostic/treatment device which incorporates pressure curves and cardiac rhythm. Further analytics as discussed above with respect to any of the systems 100, 200, 300, 600, and/or 700 may then be applied. For example, the processor 1012 may be able to diagnose, predict, and/or provide treatment for the physiological status 1038 of a patient from an algorithmic application stored, for example, within the memory 1014. Alternatively or in addition, one or more machine learning models may be used for diagnostic and/or predictive purposes. Moreover, this diagnostic function of the diagnostic/treatment device 1010 can be implemented therapeutically. For example, by determining respiratory rate, rhythm changes, respiratory movement, respiratory function, pressure changes, fluid status, chronic cardiac function changes, low ejection fraction, preserved ejection fraction, and mediastinal diseases, the data can influence the timing of operation of an ICD or other medical device designed to treat the heart or other organs of the patient. This can allow for improved monitoring (and/or improved specificity of the collected or measured data), which in turn, can reduce the likelihood of false positive diagnoses as well as false positives in therapeutic/treatment decision-making, as described in further detail herein.

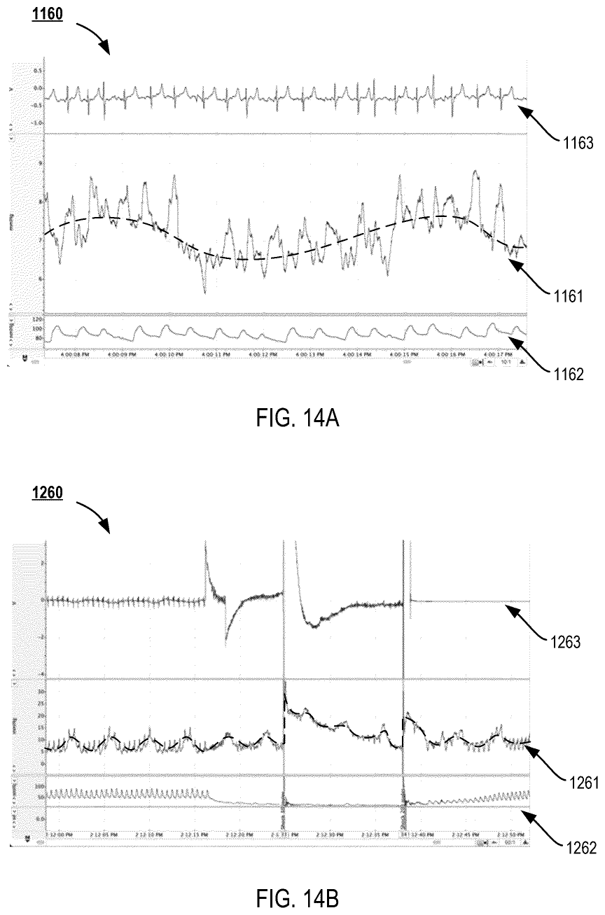

FIGS. 14A and 14B are graphs illustrating one or more relationships between electrical signal data and mediastinal pressure data. For example, as described above with reference to the diagnostic/treatment device 1010, one or more sensors (e.g., the sensors 1044) can be disposed in a patient and configured to sense, detect, monitor, and/or measure one or more bio-signals. For example, FIG. 14A is a graph 1160 showing sensor data while a patient is maintained in a normal sinus rhythm. More specifically, the graph 1160 includes pressure signal data 1161 within the mediastinum of the patient plotted over time, atrial pressure signal data 1162 plotted over time, and electrical signal data 1163 radiating from the heart of the patient and plotted over time. As shown, the pressure signal data 1161 includes and/or reflects relatively high-frequency pressure changes within the anterior mediastinum of the patient, which are associated with and/or indicative of pressure changes as a result of cardiac function. In addition, the pressure signal data 1161 includes and/or reflects relatively low-frequency pressure changes within the anterior mediastinum (e.g., shown as the dashed regression line), which are associated with and/or indicative of pressure changes as a result of respiration. The atrial pressure signal data 1162 includes and/or reflects atrial pressure changes, which can be different from and/or can have a frequency, characteristic, and/or waveform that is separate and/or different from the pressure signal data 1161 (e.g., which may reflect ventricular pressure changes). The electrical signal data 1163 includes and/or reflects changes in an electrical state of the heart. As stated above, the graph 1160 shows data collected while the patient is in a normal sinus rhythm. As such, the electrical signal data 1162 (e.g., electrocardiogram (ECG) data, cardiac electrogram data, etc.) can have a relatively normal and/or expect waveform. As shown, the changes in the pressure within the mediastinum (e.g., reflected as the pressure signal data 1161) and the changes in the atrial pressure (e.g., reflected as the atrial pressure signal data 1162) correspond with and/or are otherwise expected in light of the ECG signal data 1163.

FIG. 14B is a graph 1260 showing sensor data during an induced changed from normal sinus rhythm to ventricular fibrillation. More specifically, the graph 1260 includes pressure signal data 1261 within the mediastinum of the patient plotted over time, atrial pressure signal data 1262 plotted over time, and electrical signal data 1263 radiating from the heart of the patient and plotted over time. As shown, the pressure signal data 1261 includes and/or reflects relatively high-frequency pressure changes within the anterior mediastinum of the patient, which are associated with and/or indicative of pressure changes as a result of cardiac function (e.g., ventricular pressure changes). In addition, the pressure signal data 1261 includes and/or reflects relatively low-frequency pressure changes within the anterior mediastinum (e.g., shown as the dashed regression line), which are associated with and/or indicative of pressure changes as a result of respiration. The atrial pressure signal data 1262 includes and/or reflects atrial pressure changes, which can be different from and/or can have a frequency, characteristic, and/or waveform that is separate and/or different from the pressure signal data 1261 (e.g., which may reflect ventricular pressure changes). The electrical signal data 1263 includes and/or reflects changes in an electrical state of the heart. As stated above, the graph 1260 shows data collected during an induced changed from normal sinus rhythm to ventricular fibrillation. As such, the electrical signal data 1263 (e.g., electrocardiogram (ECG) data, cardiac electrogram data, etc.) can abruptly change from relatively normal and/or expect waveform to a waveform having a significantly higher frequency and a significantly lower amplitude. As shown, the changes in the pressure within the mediastinum (e.g., reflected as the pressure signal data 1261) and the changes in the atrial pressure (e.g., reflected as the atrial pressure signal data 1262) correspond with and/or are otherwise expected in light of the ECG signal data 1263. As described above, in this example, the changes in the pressure signal data 1261 and 1262 correspond to the changes in the electrical signal data associated with the heart. As such, a diagnostic/treatment device such as any of those described herein can confirm and/or verify that the cardiac mechanical (hemodynamic) state of the heart corresponds to the cardiac electrical state of the heart. In some instances, confirming and/or verifying a correlation between the cardiac mechanical (hemodynamic) state and the cardiac electrical state can reduce a likelihood of a false positive in determining a diagnostic status and/or providing, for example, shock therapy and/or treatment.

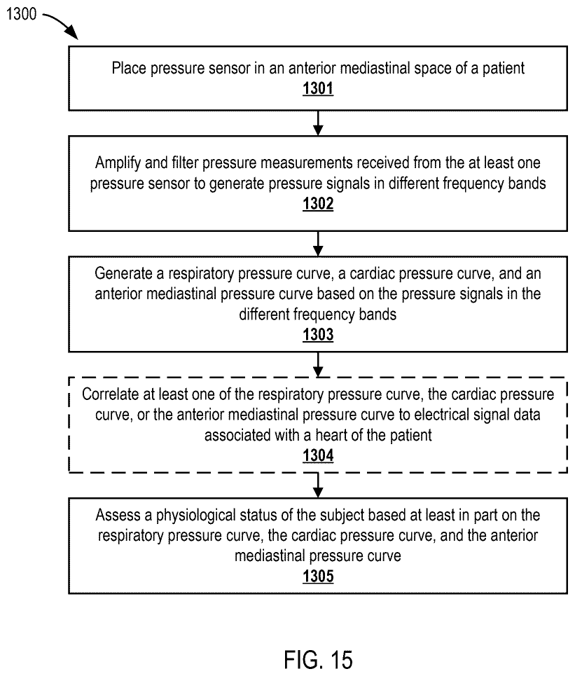

FIG. 15 is a flowchart depicting a method 1300 for assessing a physiological status of a patient dependent upon a pressure sensor, according to an embodiment. The method 1300 includes, at 1301, the placement of at least one sensor in an anterior mediastinal space of a patient. The mediastinal space is explained, for example, in the discussion of FIG. 2.

The method 1300 includes, at 1302, the amplification and filtering of received pressure measurements from the placed sensors. The amplification and/or filtering is/are performed in conjunction with example amplifier/filter(s) such as the amplifier/filter(s) of FIG. 12. The different signals are separated based on frequency band to generate pressure signals. The signals are then divided into useable information at 1303, where curves are generated based on the pressure signals in the different frequency bands. Example curves that may be generated include, but are not limited to, a cardiac hemodynamic curve, a respiratory function curve, and a basic mediastinum pressure curve. The method 1300 optionally includes, at 1304, correlating the different curves generated to and/or with electrical signal data associated with the heart of the patient, which may be optionally collected through additional sensors placed in and/or around the heart of the patient, as described in detail above with reference to specific embodiments.

The method 1300 includes, at 1305, assessing a physiological status of the patient based at least in part on the curves generated. This physiological status may be used only for diagnostic purposes, may be used for treatment purposes, or may be used in combination for diagnostic and subsequent treatment purposes, as described in detail above with reference to specific embodiments.

Some embodiments described herein relate to and/or otherwise include a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; solid state storage media such as a solid state drive (SSD) and/or a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a general-purpose processor, an FPGA, an ASIC, and/or the like. Software modules (executed on hardware) can be expressed in a variety of software languages (e.g., computer code), including C, C++, Java™, Ruby, Visual Basic™, Python™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools, and/or combinations thereof (e.g., Python™). Additional examples of computer code include, but are not limited to, control signals, encrypted code, and compressed code.

While various schematics, embodiments, and/or implementations have been described above, it should be understood that they have been presented by way of example only, and not limitation. Various modifications, changes, and/or variations in form and/or detail may be made without departing from the scope of the disclosure and/or without altering the function and/or advantages thereof unless expressly stated otherwise. Likewise, while embodiments and/or features, components, configurations, aspects, etc. thereof may be described above in the context of certain implementations, it should be understood that such implementations are presented by way of example only and not limitation. Any of the embodiments and/or features, components, configurations, aspects, etc. thereof can be used in, and/or adapted for use in, other implementations unless expressly stated otherwise. Functionally equivalent embodiments, implementations, and/or methods, in addition to those described herein, will be apparent to those skilled in the art from the foregoing descriptions and are intended to fall within the scope of the disclosure.

Where schematics, embodiments, and/or implementations described above indicate certain components arranged in certain orientations, configurations, or positions, the arrangement of components may be modified. Although various embodiments have been described as having particular features, configurations, and/or combinations of components, other embodiments are possible having a combination of any features, configurations, and/or components from any of embodiments described herein, except mutually exclusive combinations. The embodiments described herein can include various combinations and/or sub-combinations of the functions, components, configurations, and/or features of the different embodiments described. For example, the therapeutic capacities discussed above with reference to the system 200 shown in FIG. 4 can be integrated with and/or otherwise used in conjunction with the diagnostic concepts discussed above with reference to the system 700 (e.g., both diagnostic and therapeutic functionality. Additionally or alternatively, the therapeutic and diagnostic capacities described above with reference to FIG. 4 or FIG. 9 may be combined with any, all, or a combination of the amplifying, filtering, and/or diagnostic monitoring capabilities discussed above with reference to FIGS. 12-14.

The specific configurations of the various components can also be varied. For example, the size and specific shape of the various components can be different from the embodiments shown, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise.

Where methods described above indicate certain events occurring in certain order, the ordering of certain events may be modified. Additionally, certain of the events may be performed concurrently in a parallel process, when possible, as well as performed sequentially as described above. While methods have been described as having particular steps and/or combinations of steps, other methods are possible having a combination of any steps from any of methods described herein, except mutually exclusive combinations and/or unless the context clearly states otherwise.

What is claimed is:

1. A method for assessing a health status of a patient, the method comprising:
placing at least one pressure sensor in an anterior mediastinal space of the patient;
amplifying and filtering pressure signal data received from the at least one pressure sensor to separate the pressure signal data into different frequency bands;
generating a plurality of pressure curves based on the separated pressure signals in different frequency bands; and
assessing the health status of the patient based at least in part on the plurality of pressure curves.

2. The method of claim 1, wherein the plurality of pressure curves includes a respiratory pressure curve, an anterior mediastinal pressure curve, and at least one of a ventricular pressure curve or an atrial pressure curve.

3. The method of claim 1, further comprising:
receiving electrical signal data from at least one cardiac electrical sensor disposed in the anterior mediastinal space of the patient, the electrical signal data being associated with a heart of the patient.

4. The method of claim 3, further comprising:
correlating data associated with at least one of the plurality of pressure curves to the electrical signal data.

5. The method of claim 4, further comprising:
providing the data associated with at least one of the plurality of pressure curves and the electrical signal data as input into a machine learning model,
wherein the correlating the data associated with at least one of the plurality of pressure curves to the electrical signal data includes executing the machine learning model to correlate the data associated with at least one of the plurality of pressure curves to the electrical signal data.

6. The method of claim 4, wherein assessing the health status of the patient is based at least in part on the plurality of pressure curves and the electrical signal data.

7. The method of claim 1, further comprising:
generating a notification associated with the health status; and
sending, via a network, the notification to a compute device.

8. The method of claim 1, further comprising:
causing, based on the health status of the patient, a generator of an implantable cardioverter defibrillator (ICD) implanted in the patient to generate treatment energy, the ICD including a lead configured to be implanted in the anterior mediastinal space and to apply the treatment energy to a heart of the patient.

9. An apparatus for assessing a health status of a patient, the apparatus comprising:
an electrical sensor configured to be disposed within a substernal space of the patient, the electrical sensor configured to detect electrical signals radiating from a heart of the patient;
at least one pressure sensor configured to be disposed within the substernal space;
an amplifier/filter coupled to the at least one pressure sensor, the amplifier/filter configured to amplify and filter pressure signal data received from the at least one pressure sensor into pressure signals in different frequency bands; and
a processor configured to execute instructions stored in a memory that cause the processor to:
receive the pressure signals in the different frequency bands;
generate at least one pressure curve based on the pressure signals in different frequency bands;
receive, from the electrical sensor, data associated with the electrical signals radiating from the heart;
correlate the at least one pressure curve to the data associated with the electrical signals; and
determine a health status of the patient based on the correlation.

10. The apparatus of claim 9, wherein the at least one pressure curve includes a respiratory pressure curve, a mediastinal pressure curve, and at least one of a ventricular pressure curve or an atrial pressure curve.

11. The apparatus of claim 9, wherein the processor is further configured to execute instructions stored in the memory that cause the processor to:
generate a notification associated with the health status; and
send, via a network, the notification to a compute device.

12. The apparatus of claim 9, wherein the processor is further configured to execute instructions stored in the memory that cause the processor to:
provide data associated with the at least one pressure curve and data associated with electrical signals as input into a machine learning model; and
execute the machine learning model to correlate the data associated with the at least one pressure curve and data associated with electrical signals.

13. The apparatus of claim 9, wherein the apparatus is a diagnostic/treatment device, the processor further configured to execute instructions stored in the memory that cause the processor to:
control, based on the health status of the patient, a generator of an implantable cardioverter defibrillator (ICD) implanted in the patient to generate treatment energy, the ICD including a lead configured to implanted in the substernal space and to apply the treatment energy to the heart of the patient.

14. A system for reducing undesired treatments provided to a heart of a patient, the system comprising:
a lead shaped and configured to be disposed in an anterior mediastinum of a patient, the lead including an electrical sensor configured to detect electrical signals radiating from the heart of the patient, a pressure sensor configured to detect a pressure in the anterior mediastinum, and a treatment element; and
an implantable treatment device configured to be implanted in the patient and to be in communication with the lead, the implantable treatment device including a generator, a memory, and a processor configured to execute instructions stored in the memory operable to cause the processor to:
define a cardiac electrical status based on electrical signal data received from the electrical sensor;
amplify and filter pressure signal data received from the pressure sensor to separate the pressure signal data into different frequency bands; and
define a treatment decision based on a correlation between the cardiac electrical status and pressure signal data in at least one frequency band.

15. The system of claim 14, wherein the electrical signal data includes at least one of electrocardiogram data or cardiac electrogram signal data.

16. The system of claim 14, wherein the lead is sized and shaped to substantially traverse the anterior mediastinum such that a first portion of the lead is in contact with a posterior sternal surface and a second portion of the lead is in contact with the heart or is in close proximity to the heart.

17. The system of claim 14, wherein the treatment decision is a decision to provide, via the lead, treatment energy generated by the generator when a degree of the correlation between the cardiac electrical status and pressure signal data in the at least one frequency band is above a threshold degree of correlation, the treatment energy including at least one of treatment energy for cardiac pacing or treatment energy for shock therapy.

18. The system of claim 17, wherein the treatment energy for cardiac pacing is low-power treatment energy and the treatment energy for shock therapy is high-power treatment energy.

19. The system of claim 17, wherein the treatment decision is a decision to not provide treatment energy when the correlation between the cardiac electrical status and the pressure signal data in the at least one frequency band is below the threshold degree of correlation.

20. The system of claim 14, wherein the processor is further configured to execute instructions stored in the memory that cause the processor to:
generate a plurality of pressure curves based on the separated pressure signal data in the different frequency bands, the pressure signal data in the at least one frequency band being associated with a hemodynamic pressure curve.

21. The system of claim 20, wherein the plurality of pressure curves includes a respiratory pressure curve, a mediastinal pressure curve, and at least one of a ventricular pressure curve or an atrial pressure curve.

22. A non-transitory processor-readable medium storing code representing instructions to be executed by a processor, the code comprising code to cause the processor to:
receive an electrical signal from an electrical sensor of a lead implanted in an anterior mediastinum of a patient, the electrical sensor configured to detect electrical signals radiating from an external surface of a heart of a patient;
receive a pressure signal from a pressure sensor of the lead, the pressure sensor configured to detect a pressure in the anterior mediastinum;
define a suspected cardiac status based at least in part on data from the electrical signal;
confirm the cardiac status based on a correlation between the data from the electrical signal and data from the pressure signal; and
responsive to confirming the cardiac status, cause a generator of an implantable treatment device implanted in the patient to generate treatment energy, the lead configured to apply the treatment energy to the heart of the patient.

23. The non-transitory processor-readable medium of claim 22, wherein the treatment energy includes at least one of treatment energy for cardiac pacing or treatment energy for shock therapy.

24. The non-transitory processor-readable medium of claim 23, wherein the treatment energy for cardiac pacing is low-power treatment energy and the treatment energy for shock therapy is high-power treatment energy.

25. The non-transitory processor-readable medium of claim 22, wherein the electrical signal includes at least one of electrocardiogram signal data or cardiac electrogram signal data.

26. The non-transitory processor-readable medium of claim 22, wherein the data from the electrical signal is indicative of a cardiac rhythm of the heart, the suspected cardiac status being at least one of cardiac arrythmia or ventricular fibrillation.

27. The non-transitory processor-readable medium of claim 22, wherein the confirmation of the cardiac status is based on a degree of correlation between the data from the electrical signal and the data from the pressure signal.

28. The non-transitory processor-readable medium of claim 27, the code further comprising code to cause the processor to:
determine to withhold applying the treatment energy when the degree of correlation is below a threshold degree of correlation.

29. The non-transitory processor-readable medium of claim 22, the code further comprising code to cause the processor to:
amplify and filter pressure signal data received from the pressure sensor to separate the pressure signal data into different frequency bands; and
generating a plurality of pressure curves based on the separated pressure signal data in different frequency bands,
wherein the cardiac status is confirmed based on a correlation between the data from the electrical signal and at least one pressure curve from the plurality of pressure curves.

30. The non-transitory processor-readable medium of claim 29, wherein the plurality of pressure curves includes a respiratory pressure curve, a mediastinal pressure curve, and a hemodynamic pressure curve.
